(19) United States
(12) Patent Application Publication
(10) Pub. No.: US 2014/0271644 A1
(43) Pub. Date: Sep. 18, 2014

(54) COMBINATION/ADJUVANT THERAPY FOR WT-1-POSITIVE DISEASE

(71) Applicant: Memorial Sloan-Kettering Cancer Center, New York, NY (US)

(72) Inventors: David Scheinberg, New York, NY (US); Leonid Dubrovsky, New York, NY (US)

(73) Assignee: Memorial Sloan-Kettering Cancer Center, New York, NY (US)

(21) Appl. No.: 14/211,435

(22) Filed: Mar. 14, 2014

Related U.S. Application Data
(60) Provisional application No. 61/794,168, filed on Mar. 15, 2013.

(51) Int. Cl. A61K 39/395; A61K 31/506

(52) U.S. Cl. CPC A61K 39/39558; A61K 31/506
USPC 424/135.1; 424/138.1

(57) ABSTRACT

In an attempt to improve primary disease responsiveness and/or to overcome resistant disease, the present disclosure provides a method for treating or inhibiting the proliferation of a WT-1-dependent cancer comprising providing to a subject in need thereof a therapeutically effective amount of a tyrosine kinase inhibitor along with an anti-WT-1/HLA antibody, that is, an antibody that specifically binds to a peptide of Wilms' tumor protein (WT-1) presented on the surface of the cancer cells in an HLA-restricted fashion.

### CROSS-REFERENCE TO RELATED APPLICATIONS

This application contains subject matter that is related to the subject matter of commonly-assigned PCT international application serial no. PCT/US2012/031892 filed on Apr. 2, 2012 entitled “Antibodies to Cytosolic Peptides” (Docket No. 3314.013AWO), and commonly assigned, co-filed U.S. provisional application No. ______, entitled “Antibodies to Cytosolic Peptides” (Docket No. 3314.030P); the contents of each are hereby incorporated herein by reference in their entirety.

### STATEMENT OF RIGHTS UNDER FEDERALLY-SPONSORED RESEARCH

This invention was made with government support under grant NIH R01 CA 55349 and P01 CA 23766 awarded by the U.S. National Institutes of Health. The government has certain rights in the invention.

### SEQUENCE LISTING

This application contains a Sequence Listing, created on Mar. 14, 2012; the file, in ASCII format, is designated 3314031P_Sequence Listing_ST25.txt and is 177 KB. The file is hereby incorporated by reference in its entirety into the application.

### TECHNICAL FIELD

The present invention relates generally to a treatment for WT-1-positive diseases like chronic myelogenous leukemia (CML). More particularly, the invention relates to inhibition of tumor growth and combination treatment with a tyrosine kinase inhibitor therapeutic agent and antibodies against Wilm's tumor oncogene protein (WT-1).

### BACKGROUND OF THE INVENTION

To date, the treatment of cancers like CML has relied on therapeutic agents that target protein tyrosine kinase. Tyrosine kinase inhibitors (TKIs) include imatinib (GLEEVEC®) dasatinib (SPRYCEL®), sunitinib, sorafenib, pazopanib, to name a few. Tyrosine kinase inhibitors are currently the first line therapeutic in the treatment of chronic myelogenous (also referred to as myeloid or myelocytic) leukemia (CML), acute myeloid leukemia (AML), acute lymphoblastic leukemia (ALL), and myelodysplastic syndrome (MDS), ovarian cancer, prostrate cancer, soft tissue sarcoma, malignant glioma, renal cell cancer, hepatocellular carcinoma, gastrointestinal stromal tumor (GIST), breast cancer, lung cancer etc. However, the clinical efficacy of some TKIs, for example, imatinib and sunitinib, are limited by rare patient-specific intolerance to the drug or the development of treatment-refractory disease.

In addition to small molecule therapeutics that target the tyrosine kinase protein, treatments of leukemia based on immunologic approaches using vaccines and tumor-specific antibodies are being developed. For example, the Wilms' tumor oncogene protein (WT-1) has become an attractive target for immunotherapy for most leukemias, including CML, and a wide range of cancers. WT-1 is a zinc finger transcription factor that is normally expressed in mesodermal tissues during embryogenesis. In normal adult tissue, WT-1 expression is limited to low levels in CD34⁺ hematopoietic stem cells but is over-expressed in leukemias of multiple lineages and a wide range of solid tumors (1-2). More recently, WT-1 expression has been reported to be a marker of minimal residual disease. Increasing transcript levels in patients with acute myeloid leukemia (AML) in morphologic remission have been predictive of overt clinical relapse (3, 4). Furthermore, antibodies to WT-1 are detected in patients with hematopoietic malignancies and solid tumors, indicating that WT-1 is a highly immunogenic antigen (7).

For the most part, clinically approved therapeutic monoclonal antibodies (mAbs) recognize structures of cell surface proteins. WT-1, however, is a nuclear protein and, therefore, is inaccessible to classical antibody therapy. Until recently, immunotherapy targeting WT-1 had been limited to cellular approaches, exclusively aimed at generating WT-1-specific cytotoxic CD8 T cell (CTL) responses that recognize peptides presented on the cell surface by MHC class I molecules.

For induction of CTL responses, intracellular proteins are usually degraded by the proteasome or endo/lysosomes, and the resulting peptide fragments bind to MHC class I or II molecules. These peptide-MHC complexes are displayed at the cell surface where they provide targets for T cell recognition via a peptide-MHC (pMHC)-T cell receptor (TCR) interaction (8, 9). Vaccinations with peptides derived from the WT-1 protein induce HLA-restricted cytotoxic CD8 T cells, which are capable of killing tumor cells.

Other approaches to cancer treatment target cancer antigens with monoclonal antibody therapy. Monoclonal antibody (mAb) therapy has been shown to exert powerful antitumor effects by multiple mechanisms, including complement-dependent cytotoxicity (CDC), antibody-dependent cellular cytotoxicity (ADCC) and direct cell inhibition or apoptosis-inducing effects on tumor cells that over-express the target molecules.

A tremendous benefit would exist if there existed an adjuvant therapeutic approach that would improve primary disease responsiveness, overcome resistant disease, and/or lower the effective dose of an individual therapeutic agent, for example, to avoid toxicity and other adverse side effects of the TKI.

### SUMMARY OF THE INVENTION

The present disclosure provides a method for the treatment of WT-1 positive diseases based on a combination of therapeutic agents directed to different molecular targets. The approach incorporates conventional treatment with tyrosine kinase inhibitors (TKIs) such as those directed at Bcr-Abl, (imatinib and dasatinib), and TKIs directed to other molecular targets such as EGFR, for example, erlotinib and gefitinib as well as an immunotherapeutic approach based on the administration of antibodies that recognize and bind to peptides of WT-1 oncoprotein in an HLA-restricted fashion.

The present invention is based on the unexpected observation that a treatment regimen that combines a TKI and an anti-WT-1 antibody results in earlier inhibition of tumor growth and an improved anti-tumor response when compared to either administered individually. In some embodiments, co-administration of TKI with anti-WT-1 antibody permits the use of amounts of TKI that are lower than those currently utilized in treating the above-identified conditions, while maintaining the therapeutic efficacy of the TKI and moreover, while improving time-to-tumor progression, overall survival and decreasing TKI-associated side effects.

In one aspect, therefore, the invention relates to a method for treating or inhibiting the growth of a WT-1-positive cancer in a subject by administering a therapeutically effective amount of a tyrosine kinase inhibitor and a therapeutically effective amount of an isolated anti WT-1 antibody, or antigen-binding portion thereof, that is, an antibody that specifically binds to a WT-1 peptide bound to an MHC antigen. The tyrosine kinase inhibitor may be directed to a molecular target such as Bcr-Abl (imatinib, dasatinib and nilotinib), EGFR (erlotinib and gefitinib), VEGFR-1 (pazopanib and sorafenib) and others.

In one aspect, the WT-1 positive cancer is selected from the group consisting of chronic myelogenous leukemia (CML), multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma, gastrointestinal stromal tumors (GIST) and others including solid tumors.

In one aspect, the tyrosine kinase inhibitor is selected from the group consisting of imatinib, dasatinib, nilotinib, bosutinib, ponatinib, bafetinib, erlotinib, gefitinib, lapatinib, sorafenib, pazopanib and sunitinib. In one embodiment, the tyrosine kinase inhibitor is imatinib or dasatinib or a pharmaceutically acceptable salt thereof. In one embodiment, the pharmaceutically acceptable salt of imatinib is imatinib mesylate.

In another aspect, the invention relates to combination/adjuvant therapy with a TKI and an isolated anti-WT-1 antibody, or antigen-binding portion thereof. Examples of anti-WT-1 antibodies for use in combination therapy with a TKI include but are not limited to:

an anti-WT-1 antibody comprising a heavy chain (HC) variable region comprising HC-CDR1, HC-CDR2 and HC-CDR3; and a light chain (LC) variable region comprising LC-CDR1, LC-CDR2 and LC-CDR3 comprising amino acid sequences as shown in Tables 1 to 14 below and FIGS. 7-10.

In another aspect, the WT-1 antibody, or antigen-binding fragment thereof, comprises a V_{H} and V_{L} comprising first and second amino acid sequences, as shown in Tables 1 to 14 below and FIGS. 7-10. In yet another aspect, the WT-1 antibody comprises the amino acid sequence of an scFv shown in Tables 1 to 14 below and FIGS. 7-10.

The disclosed method employs a WT-1 antibody that is fully human; the antibody comprises a human variable region framework region and human constant regions. The WT-1 antibody specifically binds to a WT-1 peptide in an HLA restricted manner with a K_{D} less than 1×10⁻⁸M; in one embodiment, the K_{D} is in the range of about 1×10⁻¹¹M to about 1×10⁻⁸M. The WT-1 antibody induces antibody dependent cellular cytotoxicity (ADCC) against WT-1-positive cells.

### BRIEF DESCRIPTION OF THE DRAWINGS

FIG. 1 shows that imatinib at 1 μM, 5 μM or 10 μM does not affect antigen-dependent cellular cytotoxicity of human effector cells at various effector:target ratios (E:T) against fresh BV173 cells, a leukemia cell line derived from CML in blastic crisis (HLA-A0201⁺, Philadelphia chromosome positive). Effector to target ratios varied to demonstrate the dependence of ESKM ADCC on high E:T ratios.

FIG. 2 shows the effect of an anti-WT-1/HLA-A antibody, designated ESKM, with and without imatinib on tumor growth at intervals of 13, 20, 27, 34 and 40 days.

FIG. 3 shows luciferin imaging of BV173 xenograft NSG mice after 5 weeks of daily administration of 50 mg/kg imatinib with (lower right panel) and without (lower left panel) administration of 100 μg anti-WT-1/HLA-A antibody twice a week to mice with tumors. Control animals received neither imatinib nor antibody (upper left).

FIG. 4 shows the effects of administration of ESKM and dasatinib at 1 μM, 5 μM or 10 μM on ADCC of human effectors cells at various effector:target ratios (E:T) against BV173.

FIG. 5 shows the effect of treatment with dasatinib alone or in combination with an anti-WT-1 antibody on BV173 tumor growth in NSG mice over four weeks of treatment.

FIG. 6 shows luciferin imaging of BV173 xenograft NSG mice after five weeks of therapy. A control treatment with dasatinib alone or in combination with an anti-WT-1 antibody.

FIGS. 7-10 show amino acid sequences, including consensus sequences, for the CDRs of some embodiments of anti-WT-1 antibodies useful for the combination therapy of the disclosure.

### DETAILED DESCRIPTION OF THE INVENTION

All publications, patents and other references cited herein are incorporated by reference in their entirety into the present disclosure. Subject matter incorporated by reference is not considered to be an alternative to any claim limitations, unless otherwise explicitly indicated.

In practicing the present invention, many conventional techniques in immunology are used, which are within the skill of the ordinary artisan. These techniques are described in greater detail in, for example, “Current Protocols in Immunology” (John E. Coligan et al., eds., John Wiley & Sons, Inc. 1991 and periodic updates); Recombinant Antibodies for Immunotherapy, Melvyn Little, ed. Cambridge University Press 2009. The contents of these references and other references containing standard protocols, widely known to and relied upon by those of skill in the art, including manufacturers' instructions and dosage information are hereby incorporated by reference as part of the present disclosure. The following abbreviations are used throughout the application:

Ab: Antibody

ADCC: Antibody-dependent cellular cytotoxicity

ALL: Acute lymphocytic leukemia

AML: Acute myeloid leukemia

CDC: Complement dependent cytotoxicity

CMC: Complement mediated cytotoxicity

CDR: Complementarity determining region (see also HVR below)

C_{L}: Constant domain of the light chain

CH₁: 1^{st} constant domain of the heavy chain

CH_{1,2,3}: 1^{st}, 2^{nd} and 3^{rd} constant domains of the heavy chain

CH_{2,3}: 2^{nd} and 3^{rd} constant domains of the heavy chain

CHO: Chinese hamster ovary

CML: chronic myelogenous leukemia; also referred to as chronic myelocytic leukemia and chronic myeloid leukemia

CTL: Cytotoxic T cell

E:T Ratio: Effector:Target ratio

Fab: Antibody binding fragment

FACS: Fluorescence-activated cell sorting

FBS: Fetal bovine serum

FR: Framework region

HC: Heavy chain

HLA: Human leukocyte antigen

HVR-H: Hypervariable region-heavy chain (see also CDR)

HVR-L: Hypervariable region-light chain (see also CDR)

Ig: Immunoglobulin

K_{D}: Dissociation constant

k_{off}: Dissociation rate

kₒₙ: Association rate

MHC: Major histocompatibility complex

MM: Multiple myeloma

scFv: Single-chain variable fragment

TKI: tyrosine kinase inhibitor

V_{H}: Variable heavy chain includes heavy chain hypervariable region and heavy chain variable framework region

V_{L}: Variable light chain includes light chain hypervariable region and light chain variable framework region

WT-1: Wilms tumor protein 1

In the description that follows, terms used herein are intended to be interpreted consistently with the meaning of those terms as they are known to those of skill in the art. The definitions provided herein below are meant to clarify, but not limit, the terms defined.

As used herein, “administering” and “administration” refer to the application of an active ingredient to the body of a subject.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding portion” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” or “antigen-binding region” of an antibody, as used herein, refers to that region or portion of the antibody that binds to the antigen and which confers antigen specificity to the antibody; fragments of antigen-binding proteins, for example, antibodies includes one or more fragments of an antibody that retain the ability to specifically bind to an antigen (e.g., an peptide/HLA complex). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., 1988 Science 242:423-426; and Huston et al., 1988 Proc. Natl. Acad. Sci. 85:5879-5883. These antibody fragments are obtained using conventional techniques known to those of skill in the art, and the fragments are screened for utility in the same manner as are intact antibodies.

An “isolated antibody” is intended to encompass antibodies which have been identified and separated and/or recovered from a component of its natural environment as well as “synthetic antibodies” or “recombinant antibodies,” antibodies that are generally generated using recombinant technology or using peptide synthetic techniques known to those of skill in the art.

As used herein, the term “effective amount” means that amount of a compound or therapeutic agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The present invention provides an improved treatment method for WT-1 positive disease by the co-administration of a tyrosine kinase inhibitor and an anti-WT-1 antibody.

### Tyrosine Kinase Inhibitors

Tyrosine kinase inhibitors, as well as routes of administration and appropriate dose considerations are well known in the art for the treatment of certain cancers. These small-molecule drugs target several members of a class of proteins called tyrosine kinase enzymes that participate in signal transduction. These enzymes are overactive in some cancers, leading to uncontrolled growth.

Tyrosine kinase inhibitors suitable for use in the disclosed method include imatinib, dasatinib, nilotinib, bosutinib, ponatinib, and bafetinib imatinib, dasatinib, nilotinib, bosutinib, ponatinib, and bafetinib, erlotinib, gefitinib, lapatinib, sorafenib, and sunitinib. Table 1 provides a list of some TKIs, their molecular targets, and FDA-approved indications.

TABLE 1

| Drug | | | | |
|---|---|---|---|---|
| (Trade | | FDA-Approved | Toxicities, Side Effects | |
| name) | Target | Indications | and Precautions | Monitoring |
| Dasatinib | BCR- | Chronic myeloid | Rash; diarrhea; pleural | CBC; EKG; LFTs; |
| (Sprycel) | ABL, SRC | leukemia, acute | effusion; fluid retention; | weight; signs and |
| | family, c- | lymphocytic | mucositis; | symptoms of fluid |
| | KIT, | leukemia | myelosuppression; QT | retention |
| | PDGFR | | interval prolongation | |
| Erlotinib | EGFR | Non-small cell | Acneiform rash; diarrhea; | LFTs; signs of |
| (Tarceva) | | lung cancer, | loss of appetite; nausea | inflammatory or |
| | | pancreatic cancer | and vomiting; fatigue; | infectious sequelae in |
| | | | conjunctivitis; elevated | patients with |
| | | | LFTs | dermatologic toxicity |
| Gefitinib | EGFR | Non-small cell | Acneiform rash; diarrhea; | LFTs; signs of |
| (Iressa) | | lung cancer | loss of appetite; | inflammatory or |
| | | | interstitial lung disease | infectious sequelae in |
| | | | (rare); elevated LFTs; | patients with |
| | | | patients cannot smoke | dermatologic toxicity |
| | | | while on treatment | |
| Imatinib | BCR- | Acute | Rash; weight gain; | CBC; LFTs; weight; |
| (Gleevec) | ABL, | lymphocytic | edema; pleural effusion; | signs and symptoms |
| | c-KIT, | leukemia, chronic | cardiac toxicity | of fluid retention |
| | PDGFR | myeloid leukemia, | (depression of LVEF); | |
| | | gastrointestinal | nausea and vomiting; | |
| | | stromal tumors, | arthralgias and myalgias; | |
| | | hyperesinophilic | myelosuppression | |
| | | syndrome, | | |
| | | systemic | | |
| | | mastocytosis | | |
| Lapatinib | HER2/neu, | Breast cancer with | Cardiac toxicity | LVEF; EKG; |
| (Tykerb) | EGFR | HER2/neu | (depression of LVEF; QT | electrolyte levels; |
| | | overexpression | prolongation); acneiform | LFTs |
| | | | rash; palmar-plantar | |
| | | | erythrodysesthesia (hand- | |
| | | | foot syndrome); diarrhea; | |
| | | | nausea, vomiting and | |
| | | | dyspepsia; elevated LFTs | |
| Nilotinib | BCR- | Chronic phase or | Rash; nauseas and | CBC; LFTs; Serum |
| (Tasigna) | ABL, | accelerated Ph- | vomiting; | lipase; baseline and |
| | c-KIT, | positive CML for | myelosuppression; QTc | periodic EKGs |
| | PDGFR | patients | prolongation; sudden | |
| | | resistant/intolerant | death; electrolyte | |
| | | of prior imatinib | abnormalities; hepatic | |
| | | therapy | dysfunction; avoid in | |
| | | | patients with | |
| | | | hypokalemia, | |
| | | | hypomagnesemia, long | |
| | | | QT syndrome | |
| Sorafenib | BRAF, | Renal cell cancer, | Hypertension; alopecia; | Blood pressure; |
| (Nexavar) | VEGFR, | hepatocellular | bleeding; rash; palmar- | dermatologic toxicity |
| | EGFR, | carcinoma | plantar erythrodysesthesia | (see left); amylase, |
| | PDGFR | | (hand-foot syndrome); | lipase, and phosphate |
| | | | hypophosphatemia; | levels; CBC |
| | | | diarrhea; nausea and | |
| | | | vomiting; elevated | |
| | | | amylase and lipase levels; | |
| | | | myelosuppression; | |
| | | | wound-healing | |
| | | | complications; need to | |
| | | | discontinue treatment | |
| | | | temporarily for surgical | |
| | | | procedures | |
| Sunitinib | VEGFR, | Renal cell cancer, | Nausea and vomiting; | Adrenal function in |
| (Sutent) | PDGFR, | gastrointestinal | yellow discoloration of | patients with trauma |
| | c-KIT | stromal tumor | skin; hypothyroidism; | or severe infection, or |
| | FLT3 | | depression of LVEF; | in those undergoing |
| | | | adrenal function | surgery; blood |
| | | | abnormalities; diarrhea; | pressure; EKG; |
| | | | myelosuppression; | LVEF; CBC; |
| | | | mucositis; elevated lipase | electrolyte levels |
| | | | and creatinine levels; | (magnesium and |
| | | | elevated LFTs; increased | potassium); |
| | | | uric acid levels | phosphate levels; |
| | | | | signs and symptoms |
| | | | | of pancreatitis; |
| | | | | thyroid function tests |

Imatinib mesylate (marketed as GLEEVEC®) is approved to treat gastrointestinal stromal tumor (GIST, a rare cancer of the gastrointestinal tract) and other mesenchymal tumors, Ph⁺ CML, certain other kinds of leukemia, dermatofibrosarcoma protuberans, myelodysplastic/myeloproliferative disorders, and systemic mastocytosis. Imatinib is generally regarded as the first generation of Bcr-Abl tyrosine kinase inhibitors used for the treatment of, for example, CML. The GLEEVEC® Prescribing Information [2013: Novartis] (which is incorporated by reference in its entirety), lists recommendations for imatinib administration and relevant data.

Dasatinib (marketed as SPRYCEL®) is approved to treat some patients with CML or acute lymphoblastic leukemia. The drug is a small-molecule inhibitor of several tyrosine kinase enzymes. The SPRYCEL® Prescribing Information [Bristol-Myers Squibb] (which is incorporated by reference in its entirety), lists recommendations for dasatinib administration and relevant data.

Nilotinib (marketed as TASIGNA®) is approved to treat some patients with CML. The drug is another small-molecule tyrosine kinase inhibitor. The TASIGNA® Prescribing Information [Novartis] (which is incorporated by reference in its entirety), lists recommendations for nilotinib administration and relevant data.

Bosutinib (marketed as BOSULIF®) is also approved to treat some patients with CML and is another example of a small-molecule tyrosine kinase inhibitor. The BOSULIF® Prescribing Information [Pfizer] (which is incorporated by reference in its entirety), lists recommendations for bosutinib administration and relevant data.

Prescribing Information for each of the therapeutic agents listed in Table 1 is hereby incorporated by reference in its entirety. Additional information regarding dosing and/or adverse side effects of tyrosine kinase inhibitors can be found in G. D. Demetri, Differential properties of current tyrosine kinase inhibitors in gastrointestinal stromal tumors; Warnault P et al. Recent Advances in Drug Design of Epidermal Growth Factor Receptor Inhibitors; Sivendran S et al. Treatment-related mortality with vascular endothelial growth factor receptor tyrosine kinase inhibitor therapy in patients with advanced solid tumors: a meta-analysis; Cabezón-Gutierrez L. ALK-mutated non-small-cell lung cancer: a new strategy for cancer treatment; Barni, S. The risk for anemia with targeted therapies for solid tumor; Dasanu, C A Cardiovscular toxicity associated with small molecule tyrosine kinase inhibitors currently in clinical use. (See reference nos. 69-74 below)

### Anti-WT-1 Antibodies

The Wilms' tumor oncogene protein (WT-1) is an attractive target for immunotherapy for most leukemias and a wide range of cancers. WT-1 is a zinc finger transcription factor that is normally expressed in mesodermal tissues during embryogenesis. In normal adult tissue, WT-1 expression is limited to low levels in CD34⁺ hematopoietic stem cells but is over-expressed in leukemias of multiple lineages and a wide range of solid tumors (1-2). More recently, WT-1 expression has been reported to be a marker of minimal residual disease. Increasing transcript levels in patients with acute myeloid leukemia (AML) in morphologic remission have been predictive of overt clinical relapse (3, 4). Furthermore, antibodies to WT-1 are detected in patients with hematopoietic malignancies and solid tumors, indicating that WT-1 is a highly immunogenic antigen (7).

For the most part, clinically approved therapeutic monoclonal antibodies (mAbs) (for example, trastuzumab) recognize structures of cell surface proteins. WT-1, however, is a nuclear protein and, therefore, is inaccessible to classical antibody therapy. Until recently, immunotherapy targeting WT-1 has been limited to cellular approaches, exclusively aimed at generating WT-1-specific cytotoxic CD8 T cell (CTL) responses that recognize peptides presented on the cell surface by MHC class I molecules.

For induction of CTL responses, intracellular proteins are usually degraded by the proteasome or endo/lysosomes, and the resulting peptide fragments bind to MHC class I or II molecules. These peptide-MHC complexes are displayed at the cell surface where they provide targets for T cell recognition via a peptide-MHC (pMHC)-T cell receptor (TCR) interaction (8, 9). Vaccinations with peptides derived from the WT-1 protein induce HLA-restricted cytotoxic CD8 T cells, which are capable of killing tumor cells.

It has now been determined that co-administration of anti-WT-1 antibodies with a small molecule tyrosine kinase inhibitor can enhance the efficacy of the small molecule therapeutic.

Anti-WT-1 antibodies that may be of use for combination therapy of cancer within the scope of the claimed methods and compositions include, but are not limited to those anti-WT-1 antibodies that specifically bind a WT-1 peptide in an HLA restricted manner and further exhibit at least one of the following properties: (a) binds to WT-1/HLA with a K_{D} of about 1×10⁻¹¹ M to 1×10⁻⁸ M; (b) induces antibody dependent cellular cytotoxicity (ADCC) against WT-1-expressing cells; or (c) inhibits growth of WT-1 positive cells in vivo. In some embodiments, anti-WT-1 antibodies to be paired with TKI administration are those comprising one or more amino acid sequences (scFv, VH and VL regions or CDRs) listed in Tables 1-14 and shown in FIGS. 7-10.

TABLE 1

| Antigen | WT-1 | | |
|---|---|---|---|
| Peptide | RMFPNAPYL (SEQ ID NO: 1) | | |
| CDRs: | 1 | 2 | 3 |
| VH | GGTFSSYAIS | GIIPIFGTANYAQKFQG | RIPPYYGMDV |
| | (SEQ ID NO: 2) | (SEQ ID NO: 3) | (SEQ ID NO: 4) |
| DNA | ggaggcaccttcagcag | gggatcatccctatctttggtac | cggattcccccgtactacggtat |
| | ctatgctatcagc | agcaaactacgcacagaagttcc | ggacgtc |
| | (SEQ ID NO: 5) | agggc | (SEQ ID NO: 7) |
| | | (SEQ ID NO: 6) | |
| VL | SGSSSNIGSNYVY | RSNQRPS | AAWDDSLNGVV |
| | (SEQ ID NO: 8) | (SEQ ID NO: 9) | (SEQ ID NO: 10) |
| DNA | tctggaagcagctccaacat | aggagtaatcagcggccctca | gcagcatgggatgacagcctgaa |
| | cggaagtaattatgtatac | (SEQ ID NO: 12) | tggtgtggta |
| | (SEQ ID NO: 11) | | (SEQ ID NO: 13) |

| | |
|---|---|
| Full VH | QVQLVQSGAEVKKPGSSVKVSCKASGGTFSSYAISWVRQAPGQGLEWMGGIIPIFGTANYAQKFQGR |
| | VTITADESTSTAYMELSSLRSEDTAVYYCARRIPPYYGMDVWGQGTTVTVSS |
| | (SEQ ID NO: 14) |
| DNA | caggtgcagctggtgcagtctggggctgaggtgaagaagcctgggtcctcggtgaaggtctcctgca |
| | aggcttctggaggcaccttcagcagctatgctatcagctgggtgcgacaggcccctggacaagggct |
| | tgagtggatgggagggatcatccctatctttggtacagcaaactacgcacagaagttccagggcaga |
| | gtcacgattaccgcggacgaatccacgagcacagcctacatggagctgagcagcctgagatctgagg |
| | acacggccgtgtattactgtgcgagacggattcccccgtactacggtatggacgtctggggccaagg |
| | gaccacggtcaccgtctcctca |
| | (SEQ ID NO: 15) |
| Full VL | QTVVTQPPSASGTPGQRVTISCSGSSSNIGSNYVYWYQQLPGTAPKLLIYRSNQRPSGVPDRFSGSK |
| | SGTSASLAISGPRSVDEADYYCAAWDDSLNGVVFGGGTKLTVLG |
| | (SEQ ID NO: 16) |
| DNA | cagactgtggtgactcagccaccctcagcgtctgggacccccgggcagagggtcaccatctcttgtt |
| | ctggaagcagctccaacatcggaagtaattatgtatactggtaccaacagctcccaggaacggcccc |
| | caaactcctcatctataggagtaatcagcggccctcaggggtccctgaccgattctctggctccaag |
| | tctggcacctcagcctccctggccatcagtgggccccggtccgtggatgaggctgattattactgtg |
| | cagcatgggatgacagcctgaatggtgtggtattcggcggagggaccaagctgaccgtcctaggt |
| | (SEQ ID NO: 17) |
| scFv | QTVVTQPPSASGTPGQRVTISCSGSSSNIGSNYVYWYQQLPGTAPKLLIYRSNQRPSGVPDRFSGSK |
| | SGTSASLAISGPRSVDEADYYCAAWDDSLNGVVFGGGTKLTVLGSRGGGGSGGGGSGGGSLEMAQVQ |
| | LVQSGAEVKKPGSSVKVSCKASGGTFSSYAISWVRQAPGQGLEWMGGIIPIFGTANYAQKFQGRVTI |
| | TADESTSTAYMELSSLRSEDTAVYYCARRIPPYYGMDVWGQGTTVTVSS |
| | (SEQ ID NO: 18) |
| DNA | cagactgtggtgactcagccaccctcagcgtctgggacccccgggcagagggtcaccatctcttgtt |
| | ctggaagcagctccaacatcggaagtaattatgtatactggtaccaacagctcccaggaacggcccc |
| | caaactcctcatctataggagtaatcagcggccctcaggggtccctgaccgattctctggctccaag |
| | tctggcacctcagcctccctggccatcagtgggccccggtccgtggatgaggctgattattactgtg |
| | cagcatgggatgacagcctgaatggtgtggtattcggcggagggaccaagctgaccgtcctaggttc |
| | tagaggtggtggtggtagcggcggcggcggctctggtggtggatccctcgagatggcccaggtgcag |
| | ctggtgcagtctggggctgaggtgaagaagcctgggtcctcggtgaaggtctcctgcaaggcttctg |
| | gaggcaccttcagcagctatgctatcagctgggtgcgacaggcccctggacaagggcttgagtggat |
| | gggagggatcatccctatctttggtacagcaaactacgcacagaagttccagggcagagtcacgatt |
| | accgcggacgaatccacgagcacagcctacatggagctgagcagcctgagatctgaggacacggccg |
| | tgtattactgtgcgagacggattcccccgtactacggtatggacgtctggggccaagggaccacggt |
| | caccgtctcctca |
| | (SEQ ID NO: 19) |

TABLE 2

| Antigen | WT-1 (Ext002 #5) | | |
|---|---|---|---|
| Peptide | RMFPNAPYL (SEQ ID NO: 1) | | |
| CDRs: | 1 | 2 | 3 |
| VH | GDSVSSNSAAWN | RTYYGSKWYNDYAVSVKS | GRLGAFDI |
| | (SEQ ID NO: 20) | (SEQ ID NO: 21) | (SEQ ID NO: 22) |
| DNA | ggggacagtgtctctagc | aggacatactacgggtccaag | ggtcgcttaggggatgcttttga |
| | aacagtgctgcttggaac | tggtataatgattatgcagta | tatc |
| | (SEQ ID NO: 23) | tctgtgaaaagt | (SEQ ID NO: 25) |
| | | (SEQ ID NO: 24) | |
| VL | RASQSISSYLN | AASSLQS | QQSYSTPLT |
| | (SEQ ID NO: 26) | (SEQ ID NO: 27) | (SEQ ID NO: 28) |
| DNA | cgggcaagtcagagcatt | gctgcatccagtttgcaaagt | caacagagttacagtacccctct |
| | agcagctatttaaat | (SEQ ID NO: 30) | cact |
| | (SEQ ID NO: 29) | | (SEQ ID NO: 31) |

| | |
|---|---|
| Full VH | QVQLQQSGPGLVKPSQTLSLTCAISGDSVSSNSAAWNWIRQSPSRGLEWLGRTYYGSKWYNDYAV |
| | SVKSRITINPDTSKNQFSLQLNSVTPEDTAVYYCARGRLGDAFDIWGQGTMVTVSS |
| | (SEQ ID NO: 32) |
| DNA | caggtacagctgcagcagtcaggtccaggactggtgaagccctcgcagaccctctcactcacctg |
| | tgccatctccggggacagtgtctctagcaacagtgctgcttggaactggatcaggcagtccccat |
| | cgagaggccttgagtggctgggaaggacatactacgggtccaagtggtataatgattatgcagta |
| | tctgtgaaaagtcgaataaccatcaacccagacacatccaagaaccagttctccctgcagctgaa |
| | ctctgtgactcccgaggacacggctgtgtattactgtgcaagaggtcgcttaggggatgcttttg |
| | atatctggggccaagggacaatggtcaccgtctcttca |
| | (SEQ ID NO: 33) |
| Full VL | DIQMTQSPSSLSASVGDRVTITCRASQSISSYLNWYQQKPGKAPKLLIYAASSLQSGVPSRFSGS |
| | GSGTDFTLTISSLQPEDFATYYCQQSYSTPLTFGGGTKVDIKR |
| | (SEQ ID NO: 34) |
| DNA | gacatccagatgacccagtctccatcctccctgtctgcatctgtaggagacagagtcaccatcac |
| | ttgccgggcaagtcagagcattagcagctatttaaattggtatcagcagaaaccagggaaagccc |
| | ctaagctcctgatctatgctgcatccagtttgcaaagtggggtcccatcaaggttcagtggcagt |
| | ggatctgggacagatttcactctcaccatcagcagtctgcaacctgaagattttgcaacttacta |
| | ctgtcaacagagttacagtacccctctcactttcggcggagggaccaaagtggatatcaaacgt |
| | (SEQ ID NO: 35) |
| scFv | DIQMTQSPSSLSASVGDRVTITCRASQSISSYLNWYQQKPGKAPKLLIYAASSLQSGVPSRFSGS |
| | GSGTDFTLTISSLQPEDFATYYCQQSYSTPLTFGGGTKVDIKRSRGGGGSGGGGSGGGGSLEMAQ |
| | VQLQQSGPGLVKPSQTLSLTCAISGDSVSSNSAAWNWIRQSPSRGLEWLGRTYYGSKWYNDYAVS |
| | VKSRITINPDTSKNQFSLQLNSVTPEDTAVYYCARGRLGDAFDIWGQGTMVTVSS |
| | (SEQ ID NO: 36) |
| DNA | gacatccagatgacccagtctccatcctccctgtctgcatctgtaggagacagagtcaccatcac |
| | ttgccgggcaagtcagagcattagcagctatttaaattggtatcagcagaaaccagggaaagccc |
| | ctaagctcctgatctatgctgcatccagtttgcaaagtggggtcccatcaaggttcagtggcagt |
| | ggatctgggacagatttcactctcaccatcagcagtctgcaacctgaagattttgcaacttacta |
| | ctgtcaacagagttacagtacccctctcactttcggcggagggaccaaagtggatatcaaacgtt |
| | ctagaggtggtggtggtagcggcggcggcggctctggtggtggtggatccctcgagatggcccag |
| | gtacagctgcagcagtcaggtccaggactggtgaagccctcgcagaccctctcactcacctgtgc |
| | catctccggggacagtgtctctagcaacagtgctgcttggaactggatcaggcagtccccatcga |
| | gaggccttgagtggctgggaaggacatactacgggtccaagtggtataatgattatgcagtatct |
| | gtgaaaagtcgaataaccatcaacccagacacatccaagaaccagttctccctgcagctgaactc |
| | tgtgactcccgaggacacggctgtgtattactgtgcaagaggtcgcttaggggatgcttttgata |
| | tctggggccaagggacaatggtcaccgtctcttca |
| | (SEQ ID NO: 37) |

TABLE 3

| Antigen | WT-1 (Ext002 #13) | | |
|---|---|---|---|
| Peptide | RMFPNAPYL (SEQ ID NO: 1) | | |
| CDRs: | 1 | 2 | 3 |
| VH | GYSFTNFWIS | RVDPGYSYSTYSPSFQG | VQYSGYYDWFDP |
| | (SEQ ID NO: 38) | (SEQ ID NO: 39) | (SEQ ID NO: 40) |
| DNA | ggatacagcttcaccaactt | agggttgatcctggctactctta | gtacaatatagtggctactatg |
| | ctggatcagc | tagcacctacagcccgtccttcc | actggttcgacccc |
| | (SEQ ID NO: 41) | aaggc | (SEQ ID NO: 43) |
| | | (SEQ ID NO: 42) | |
| VL | SGSSSNIGSNTVN | SNNQRPS | AAWDDSLNGWN |
| | (SEQ ID NO: 44) | (SEQ ID NO: 45) | (SEQ ID NO: 46) |
| DNA | tctggaagcagctccaacat | agtaataatcagcggccctca | gcagcatgggatgacagcctga |
| | cggaagtaatactgtaaac | (SEQ ID NO: 48) | atggttgggtg |
| | (SEQ ID NO: 47) | | (SEQ ID NO: 49) |

| | |
|---|---|
| Full VH | QMQLVQSGAEVKEPGESLRISCKGSGYSFTNFWISWVRQMPGKGLEWMGRVDPGYSYSTYSPSFQG |
| | HVTISADKSTSTAYLQWNSLKASDTAMYYCARVQYSGYYDWFDPWGQGTLVTVSS |
| | (SEQ ID NO: 50) |
| DNA | cagatgcagctggtgcagtccggagcagaggtgaaagagcccggggagtctctgaggatctcctgt |
| | aagggttctggatacagcttcaccaacttctggatcagctgggtgcgccagatgcccgggaaaggc |
| | ctggagtggatggggagggttgatcctggctactcttatagcacctacagcccgtccttccaaggc |
| | cacgtcaccatctcagctgacaagtctaccagcactgcctacctgcagtggaacagcctgaaggcc |
| | tcggacaccgccatgtattactgtgcgagagtacaatatagtggctactatgactggttcgacccc |
| | tggggccagggaaccctggtcaccgtctcctca |
| | (SEQ ID NO: 51) |
| Full VL | QAVVTQPPSASGTPGQRVTISCSGSSSNIGSNTVNWYQQVPGTAPKLLIYSNNQRPSGVPDRFSGS |
| | KSGTSASLAISGLQSEDEADYYCAAWDDSLNGWVFGGGTKLTVLG |
| | (SEQ ID NO: 52) |
| DNA | caggctgtggtgactcagccaccctcagcgtctgggacccccgggcagagggtcaccatctcttgt |
| | tctggaagcagctccaacatcggaagtaatactgtaaactggtaccagcaggtcccaggaacggcc |
| | cccaaactcctcatctatagtaataatcagcggccctcaggggtccctgaccgattctctggctcc |
| | aagtctggcacctcagcctccctggccatcagtgggctccagtctgaggatgaggctgattattac |
| | tgtgcagcatgggatgacagcctgaatggttgggtgttcggcggagggaccaagctgaccgtccta |
| | ggt |
| | (SEQ ID NO: 53) |
| scFv | QAVVTQPPSASGTPGQRVTISCSGSSSNIGSNTVNWYQQVPGTAPKLLIYSNNQRPSGVPDRFSGS |
| | KSGTSASLAISGLQSEDEADYYCAAWDDSLNGWVFGGGTKLTVLGSRGGGGSGGGGSGGGGSLEMA |
| | QMQLVQSGAEVKEPGESLRISCKGSGYSFTNFWISWVRQMPGKGLEWMGRVDPGYSYSTYSPSFQG |
| | HVTISADKSTSTAYLQWNSLKASDTAMYYCARVQYSGYYDWFDPWGQGTLVTVSS |
| | (SEQ ID NO: 54) |
| DNA | caggctgtggtgactcagccaccctcagcgtctgggacccccgggcagagggtcaccatctcttgt |
| | tctggaagcagctccaacatcggaagtaatactgtaaactggtaccagcaggtcccaggaacggcc |
| | cccaaactcctcatctatagtaataatcagcggccctcaggggtccctgaccgattctctggctcc |
| | aagtctggcacctcagcctccctggccatcagtgggctccagtctgaggatgaggctgattattac |
| | tgtgcagcatgggatgacagcctgaatggttgggtgttcggcggagggaccaagctgaccgtccta |
| | ggttctagaggtggtggtggtagcggcggcggcggctctggtggtggtggatccctcgagatggcc |
| | cagatgcagctggtgcagtccggagcagaggtgaaagagcccggggagtctctgaggatctcctgt |
| | aagggttctggatacagcttcaccaacttctggatcagctgggtgcgccagatgcccgggaaaggc |
| | ctggagtggatggggagggttgatcctggctactcttatagcacctacagcccgtccttccaaggc |
| | cacgtcaccatctcagctgacaagtctaccagcactgcctacctgcagtggaacagcctgaaggcc |
| | tcggacaccgccatgtattactgtgcgagagtacaatatagtggctactatgactggttcgacccc |
| | tggggccagggaaccctggtcaccgtctcctca |
| | (SEQ ID NO: 55) |

TABLE 4

| Antigen | WT-1 (Ext002 #15) | | |
|---|---|---|---|
| Peptide | RMFPNAPYL (SEQ ID NO: 1) | | |
| CDRs: | 1 | 2 | 3 |
| VH | GYNFSNKWIG | IIYPGYSDITYSPSFQG | HTALAGFDY |
| | (SEQ ID NO: 56) | (SEQ ID NO: 57) | (SEQ ID NO: 58) |
| DNA | ggctacaactttagcaaca | atcatctatcccggttactcgg | cacacagctttggccggctttg |
| | agtggatcggc | acatcacctacagcccgtcctt | actac |
| | (SEQ ID NO: 59) | ccaaggc | (SEQ ID NO: 61) |
| | | (SEQ ID NO: 60) | |
| VL | RASQNINKWLA | KASSLES | QQYNSYAT |
| | (SEQ ID NO: 62) | (SEQ ID NO: 63) | (SEQ ID NO: 64) |
| DNA | Cgggccagtcagaatatc | aaggcgtctagtttagaaagt | caacaatataatagttatgcga |
| | aataagtggctggcc | (SEQ ID NO: 66) | cg |
| | (SEQ ID NO: 65) | | (SEQ ID NO: 67) |

| | |
|---|---|
| Full VH | QVQLVQSGAEVKKPGESLKISCKGSGYNFSNKWIGWVRQLPGRGLEWIAIIYPGYSDITYSPSFQGRV |
| | TISADTSINTAYLHWHSLKASDTAMYYCVRHTALAGFDYWGLGTLVTVSS |
| | (SEQ ID NO: 68) |
| DNA | caggtgcagctggtgcagtctggagcagaggtgaaaaagcccggagagtctctgaagatctcctgtaa |
| | gggttctggctacaactttagcaacaagtggatcggctgggtgcgccaattgcccgggagaggcctgg |
| | agtggatagcaatcatctatcccggttactcggacatcacctacagcccgtccttccaaggccgcgtc |
| | accatctccgccgacacgtccattaacaccgcctacctgcactggcacagcctgaaggcctcggacac |
| | cgccatgtattattgtgtgcgacacacagctttggccggctttgactactggggcctgggcaccctgg |
| | tcaccgtctcctca |
| | (SEQ ID NO: 69) |
| Full VL | DIQMTQSPSTLSASVGDRVTITCRASQNINKWLAWYQQRPGKAPQLLIYKASSLESGVPSRFSGSGSG |
| | TEYTLTISSLQPDDFATYYCQQYNSYATFGQGTKVEIKR |
| | (SEQ ID NO: 70) |
| DNA | gacatccagatgacccagtctccttccaccctgtctgcatctgtaggagacagagtcacaatcacttg |
| | ccgggccagtcagaatatcaataagtggctggcctggtatcagcagagaccagggaaagcccctcagc |
| | tcctgatctataaggcgtctagtttagaaagtggggtcccatctaggttcagcggcagtggatctggg |
| | acagaatacactctcaccatcagcagcctgcagcctgatgattttgcaacttattactgccaacaata |
| | taatagttatgcgacgttcggccaagggaccaaggtggaaatcaaacgt |
| | (SEQ ID NO: 71) |
| scFv | DIQMTQSPSTLSASVGDRVTITCRASQNINKWLAWYQQRPGKAPQLLIYKASSLESGVPSRFSGSGSG |
| | TEYTLTISSLQPDDFATYYCQQYNSYATFGQGTKVEIKRSRGGGGSGGGGSGGGGSLEMAQVQLVQSG |
| | AEVKKPGESLKISCKGSGYNFSNKWIGWVRQLPGRGLEWIAIIYPGYSDITYSPSFQGRVTISADTSI |
| | NTAYLHWHSLKASDTAMYYCVRHTALAGFDYWGLGTLVTVSS |
| | (SEQ ID NO: 72) |
| DNA | gacatccagatgacccagtctccttccaccctgtctgcatctgtaggagacagagtcacaatcacttg |
| | ccgggccagtcagaatatcaataagtggctggcctggtatcagcagagaccagggaaagcccctcagc |
| | tcctgatctataaggcgtctagtttagaaagtggggtcccatctaggttcagcggcagtggatctggg |
| | acagaatacactctcaccatcagcagcctgcagcctgatgattttgcaacttattactgccaacaata |
| | taatagttatgcgacgttcggccaagggaccaaggtggaaatcaaacgttctagaggtggtggtggta |
| | gcggcggcggcggctctggtggtggtggatccctcgagatggcccaggtgcagctggtgcagtctgga |
| | gcagaggtgaaaaagcccggagagtctctgaagatctcctgtaagggttctggctacaactttagcaa |
| | caagtggatcggctgggtgcgccaattgcccgggagaggcctggagtggatagcaatcatctatcccg |
| | gttactcggacatcacctacagcccgtccttccaaggccgcgtcaccatctccgccgacacgtccatt |
| | aacaccgcctacctgcactggcacagcctgaaggcctcggacaccgccatgtattattgtgtgcgaca |
| | cacagctttggccggctttgactactggggcctgggcaccctggtcaccgtctcctca |
| | (SEQ ID NO: 73) |

TABLE 5

| Antigen | WT-1 (Ext002 #18) | | |
|---|---|---|---|
| Peptide | RMFPNAPYL (SEQ ID NO: 1) | | |
| CDRs: | 1 | 2 | 3 |
| VH | GFTFDDYGMS | GINWNGGSTGYADSVRG | ERGYGYHDPHDY |
| | (SEQ ID NO: 74) | (SEQ ID NO: 75) | (SEQ ID NO: 76) |
| DNA | gggttcacctttgatgattat | ggtattaattggaatggtggt | gagcgtggctacgggtaccat |
| | ggcatgagc | agcacaggttatgcagactc | gatccccatgactac |
| | (SEQ ID NO: 77) | tgtgaggggc | (SEQ ID NO: 79) |
| | | (SEQ ID NO: 78) | |
| VL | GRNNIGSKSVH | DDSDRPS | QVWDSSSDHVV |
| | (SEQ ID NO: 80) | (SEQ ID NO: 81) | (SEQ ID NO: 82) |
| DNA | gggagaaacaacattggaagt | gatgatagcgaccggccctca | caggtgtgggatagtagtagt |
| | aaaagtgtgcac | (SEQ ID NO: 84) | gatcatgtggta |
| | (SEQ ID NO: 83) | | (SEQ ID NO: 85) |

| | |
|---|---|
| Full VH | EVQLVQSGGGVVRPGGSLRLSCAASGFTFDDYGMSWVRQAPGKGLEWVSGINWNGGSTGYADSVRG |
| | RFTISRDNAKNSLYLQMNSLRAEDTALYYCARERGYGYHDPHDYWGQGTLVTVSS |
| | (SEQ ID NO: 86) |
| DNA | gaagtgcagctggtgcagtctgggggaggtgtggtacggcctggggggtccctgagactctcctgt |
| | gcagcctctgggttcacctttgatgattatggcatgagctgggtccgccaagctccagggaagggg |
| | ctggagtgggtctctggtattaattggaatggtggtagcacaggttatgcagactctgtgaggggc |
| | cgattcaccatctccagagacaacgccaagaactccctgtatctgcaaatgaacagtctgagagcc |
| | gaggacacggccttgtattactgtgcgagagagcgtggctacgggtaccatgatccccatgactac |
| | tggggccaaggcaccctggtgaccgtctcctca |
| | (SEQ ID NO: 87) |
| Full VL | QSVVTQPPSVSVAPGKTARITCGRNNIGSKSVHWYQQKPGQAPVLVVYDDSDRPSGIPERFSGSNS |
| | GNTATLTISRVEAGDEADYYCQVWDSSSDHVVFGGGTKLTVLG |
| | (SEQ ID NO: 88) |
| DNA | cagtctgtcgtgacgcagccgccctcggtgtcagtggccccaggaaagacggccaggattacctgt |
| | gggagaaacaacattggaagtaaaagtgtgcactggtaccagcagaagccaggccaggcccctgtg |
| | ctggtcgtctatgatgatagcgaccggccctcagggatccctgagcgattctctggctccaactct |
| | gggaacacggccaccctgaccatcagcagggtcgaagccggggatgaggccgactattactgtcag |
| | gtgtgggatagtagtagtgatcatgtggtattcggcggagggaccaagctgaccgtcctaggt |
| | (SEQ ID NO: 89) |
| scFv | QSVVTQPPSVSVAPGKTARITCGRNNIGSKSVHWYQQKPGQAPVLVVYDDSDRPSGIPERFSGSNS |
| | GNTATLTISRVEAGDEADYYCQVWDSSSDHVVFGGGTKLTVLGSRGGGGSGGGGSGGSLEMAEVQL |
| | VQSGGGVVRPGGSLRLSCAASGFTFDDYGMSWVRQAPGKGLEWVSGINWNGGSTGYADSVRGRFTI |
| | SRDNAKNSLYLQMNSLRAEDTALYYCARERGYGYHDPHDYWGQGTLVTVSS |
| | (SEQ ID NO: 90) |
| DNA | cagtctgtcgtgacgcagccgccctcggtgtcagtggccccaggaaagacggccaggattacctgt |
| | gggagaaacaacattggaagtaaaagtgtgcactggtaccagcagaagccaggccaggcccctgtg |
| | ctggtcgtctatgatgatagcgaccggccctcagggatccctgagcgattctctggctccaactct |
| | gggaacacggccaccctgaccatcagcagggtcgaagccggggatgaggccgactattactgtcag |
| | gtgtgggatagtagtagtgatcatgtggtattcggcggagggaccaagctgaccgtcctaggttct |
| | agaggtggtggtggtagcggcggcggcggctctggtggatccctcgagatggccgaagtgcagctg |
| | gtgcagtctgggggaggtgtggtacggcctggggggtccctgagactctcctgtgcagcctctggg |
| | ttcacctttgatgattatggcatgagctgggtccgccaagctccagggaaggggctggagtgggtc |
| | tctggtattaattggaatggtggtagcacaggttatgcagactctgtgaggggccgattcaccatc |
| | tccagagacaacgccaagaactccctgtatctgcaaatgaacagtctgagagccgaggacacggcc |
| | ttgtattactgtgcgagagagcgtggctacgggtaccatgatccccatgactactggggccaaggc |
| | accctggtgaccgtctcctca |
| | (SEQ ID NO: 91) |

TABLE 6

| Antigen | WT-1 (Ext002 #23) | | |
|---|---|---|---|
| Peptide | RMFPNAPYL (SEQ ID NO. 1) | | |
| CDRs: | 1 | 2 | 3 |
| VH | GFSVSGTYMG | LLYSGGGTYHPASLQG | GGAGGGHFDS |
| | (SEQ ID NO. 92) | (SEQ ID NO. 93) | (SEQ ID NO. 94) |
| DNA | gggttctccgtcagtggcacc | cttctttatagtggtggcggcac | gaggggcaggaggtggccac |
| | tacatgggc | ataccacccagcgtccctgcagg | tttgactcc |
| | (SEQ ID NO. 95) | gc | (SEQ ID NO. 97) |
| | | (SEQ ID NO. 96) | |
| VL | TGSSSNIGAGYDVH | GNSNRPS | AAWDDSLNGYV |
| | (SEQ ID NO. 98) | (SEQ ID NO. 99) | (SEQ ID NO. 100) |
| DNA | actgggagcagctccaacatc | ggtaacagcaatcggccctca | gcagcatgggatgacagcct |
| | ggggcaggttatgatgtacac | (SEQ ID NO. 102) | gaatggttatgtc |
| | (SEQ ID NO. 101) | | (SEQ ID NO. 103) |

| | |
|---|---|
| Full VH | EVQLVETGGGLLQPGGSLRLSCAASGFSVSGTYMGWVRQAPGKGLEWVALLYSGGGTYHPASLQGR |
| | FIVSRDSSKNMVYLQMNSLKAEDTAVYYCAKGGAGGGHFDSWGQGTLVTVSS |
| | (SEQ ID NO. 104) |
| DNA | gaggtgcagctggtggagaccggaggaggcttgctccagccgggggggtccctcagactctcctgt |
| | gcagcctctgggttctccgtcagtggcacctacatgggctgggtccgccaggctccagggaaggga |
| | ctggagtgggtcgcacttctttatagtggtggcggcacataccacccagcgtccctgcagggccga |
| | ttcatcgtctccagagacagctccaagaatatggtctatcttcaaatgaatagcctgaaagccgag |
| | gacacggccgtctattactgtgcgaaaggaggggcaggaggtggccactttgactcctggggccaa |
| | ggcaccctggtgaccgtctcctca |
| | (SEQ ID NO. 105) |
| Full VL | QSVLTQPPSVSGAPGQRVTISCTGSSSNIGAGYDVHWYQQLPGTAPKLLIYGNSNRPSGVPDRFSG |
| | SKSGTSASLAISGLQSEDEADYYCAAWDDSLNGYVFGTGTKLTVLG |
| | (SEQ ID NO. 106) |
| DNA | cagtctgtgttgacgcagccgccctcagtgtctggggccccagggcagagggtcaccatctcctgc |
| | actgggagcagctccaacatcggggcaggttatgatgtacactggtaccagcagcttccaggaaca |
| | gcccccaaactcctcatctatggtaacagcaatcggccctcaggggtccctgaccgattctctggc |
| | tccaagtctggcacctcagcctccctggccatcagtgggctccagtctgaggatgaggctgattat |
| | tactgtgcagcatgggatgacagcctgaatggttatgtcttcggaactgggaccaagctgaccgtc |
| | ctaggt |
| | (SEQ ID NO. 107) |
| scFv | QSVLTQPPSVSGAPGQRVTISCTGSSSNIGAGYDVHWYQQLPGTAPKLLIYGNSNRPSGVPDRFSG |
| | SKSGTSASLAISGLQSEDEADYYCAAWDDSLNGYVFGTGTKLTVLGSRGGGGSGGGGSGGGGSLEM |
| | AEVQLVETGGGLLQPGGSLRLSCAASGFSVSGTYMGWVRQAPGKGLEWVALLYSGGGTYHPASLQG |
| | RFIVSRDSSKNMVYLQMNSLKAEDTAVYYCAKGGAGGGHFDSWGQGTLVTVSS |
| | (SEQ ID NO. 108) |
| DNA | cagtctgtgttgacgcagccgccctcagtgtctggggccccagggcagagggtcaccatctcctgc |
| | actgggagcagctccaacatcggggcaggttatgatgtacactggtaccagcagcttccaggaaca |
| | gcccccaaactcctcatctatggtaacagcaatcggccctcaggggtccctgaccgattctctggc |
| | tccaagtctggcacctcagcctccctggccatcagtgggctccagtctgaggatgaggctgattat |
| | tactgtgcagcatgggatgacagcctgaatggttatgtcttcggaactgggaccaagctgaccgtc |
| | ctaggttctagaggtggtggtggtagcggcggcggcggctctggtggtggtggatccctcgagatg |
| | gccgaggtgcagctggtggagaccggaggaggcttgctccagccgggggggtccctcagactctcc |
| | tgtgcagcctctgggttctccgtcagtggcacctacatgggctgggtccgccaggctccagggaag |
| | ggactggagtgggtcgcacttctttatagtggtggcggcacataccacccagcgtccctgcagggc |
| | cgattcatcgtctccagagacagctccaagaatatggtctatcttcaaatgaatagcctgaaagcc |
| | gaggacacggccgtctattactgtgcgaaaggaggggcaggaggtggccactttgactcctggggc |
| | caaggcaccctggtgaccgtctcctca |
| | (SEQ ID NO. 109) |

TABLE 7

| Antigen | WT-1 (Ext002B #17) | | |
|---|---|---|---|
| Peptide | CMTWNCHNL (SEQ ID NO: 239) | | |
| CDRs: | 1 | 2 | 3 |
| VH | GYTLTELSMH | GFDPEDGETIYAQKFQG | SFYSYYGIDT |
| | (SEQ ID NO: 240) | (SEQ ID NO: 241) | (SEQ ID NO: 242) |
| DNA | ggatacaccctcactgaattat | ggttttgatcctgaagatggtgaa | tctttctactcttactacggt |
| | ccatgcac | acaatctacgcacagaagttccag | atcgatact |
| | (SEQ ID NO: 243) | ggc | (SEQ ID NO: 245) |
| | | (SEQ ID NO: 244) | |
| VL | QGDSLRRYYAS | ANNNRPS | NSRDISVNGWM |
| | (SEQ ID NO: 246) | (SEQ ID NO: 247) | (SEQ ID NO: 248) |
| DNA | caaggagacagcctcagaaggt | gctaataacaatcggccctca | aactcccgggacatcagtgtt |
| | attatgcaagc | (SEQ ID NO: 250) | aacggttggatg |
| | (SEQ ID NO: 249) | | (SEQ ID NO: 251) |

| | |
|---|---|
| Full VH | QVQLVQSGAEVKKPGASVKVSCKVSGYTLTELSMHWVRQAPGKGLEWMGGFDPEDGETIYAQKFQGRVTM |
| | TEDTSTDTAYMELSSLRSEDTAVYYCARSFYSYYGIDTWGQGTLVTVSS |
| | (SEQ ID NO: 252) |
| DNA | caggtgcagctggtgcagtctggggctgaggtgaagaagcctggggcctcagtgaaggtctcctgcaagg |
| | tttccggatacaccctcactgaattatccatgcactgggtgcggcaggctcctggaaaagggcttgagtg |
| | gatgggaggttttgatcctgaagatggtgaaacaatctacgcacagaagttccagggcagagtcaccatg |
| | accgaggacacatctacagacacagcctacatggagctgagcagcctgagatctgaggacacggccgtgt |
| | attactgtgcgcgctctttctactcttactacggtatcgatacttggggtcaaggtactctggtgaccgt |
| | ctcctca |
| | (SEQ ID NO: 253) |
| Full VL | SSELTQDPAVSVALGQTVRITCQGDSLRRYYASWYQQKPGQAPVLVIYANNNRPSGIPDRFSGSSSGNTA |
| | SLTITGAQAEDEADYYCNSRDISVNGWMFGGGTKLTVLG |
| | (SEQ ID NO: 254) |
| DNA | tcttctgagctgactcaggaccctgctgtgtctgtggccttgggacagacagtcaggatcacatgccaag |
| | gagacagcctcagaaggtattatgcaagctggtaccagcagaagccaggacaggcccctgtacttgtcat |
| | ctatgctaataacaatcggccctcagggatcccagaccgattctctggctccagctcaggaaacacagct |
| | tccttgaccatcactggggctcaggcggaggatgaggctgactattattgtaactcccgggacatcagtg |
| | ttaacggttggatgttcggcggagggaccaagctgaccgtcctaggt |
| | (SEQ ID NO: 255) |
| scFv | SSELTQDPAVSVALGQTVRITCQGDSLRRYYASWYQQKPGQAPVLVIYANNNRPSGIPDRFSGSSSGNTA |
| | SLTITGAQAEDEADYYCNSRDISVNGWMFGGGTKLTVLGSRGGGGSGGGGSGGGGSLEMAQVQLVQSGAE |
| | VKKPGASVKVSCKVSGYTLTELSMHWVRQAPGKGLEWMGGFDPEDGETIYAQKFQGRVTMTEDTSTDTAY |
| | MELSSLRSEDTAVYYCARSFYSYYGIDTWGQGTLVTVSS |
| | (SEQ ID NO: 256) |
| DNA | tcttctgagctgactcaggaccctgctgtgtctgtggccttgggacagacagtcaggatcacatgccaag |
| | gagacagcctcagaaggtattatgcaagctggtaccagcagaagccaggacaggcccctgtacttgtcat |
| | ctatgctaataacaatcggccctcagggatcccagaccgattctctggctccagctcaggaaacacagct |
| | tccttgaccatcactggggctcaggcggaggatgaggctgactattattgtaactcccgggacatcagtg |
| | ttaacggttggatgttcggcggagggaccaagctgaccgtcctaggttctagaggtggtggtggtagcgg |
| | cggcggcggctctggtggtggtggatccctcgagatggcccaggtgcagctggtgcagtctggggctgag |
| | gtgaagaagcctggggcctcagtgaaggtctcctgcaaggtttccggatacaccctcactgaattatcca |
| | tgcactgggtgcggcaggctcctggaaaagggcttgagtggatgggaggttttgatcctgaagatggtga |
| | aacaatctacgcacagaagttccagggcagagtcaccatgaccgaggacacatctacagacacagcctac |
| | atggagctgagcagcctgagatctgaggacacggccgtgtattactgtgcgcgctctttctactcttact |
| | acggtatcgatacttggggtcaaggtactctggtgaccgtctcctca |
| | (SEQ ID NO: 257) |

TABLE 8

| Antigen | WT-1 (Ext002B #28) | | |
|---|---|---|---|
| Peptide | CMTWNQMNL (SEQ ID NO: 239) | | |
| CDRs: | 1 | 2 | 3 |
| VH | GYTFTTYGMN | WINTNTGKPTYAQGFTG | GYYGWDYHDY |
| | (SEQ ID NO: 258) | (SEQ ID NO: 259) | (SEQ ID NO: 260) |
| DNA | ggatacaccttcactacctatgg | tggatcaacaccaacactgggaa | ggttactacggttgggactacca |
| | tatgaat | gccaacgtatgcccagggcttca | tgattac |
| | (SEQ ID NO: 261) | cagga | (SEQ ID NO: 263) |
| | | (SEQ ID NO: 262) | |
| VL | GGNNIGSKSVH | YDSDRPS | QVWDSSSDHSPYV |
| | (SEQ ID NO: 264) | (SEQ ID NO: 265) | (SEQ ID NO: 266) |
| DNA | gggggaaacaacattggaagtaa | tatgatagcgaccggccctca | caggtgtgggatagtagtagtga |
| | aagtgtgcac | (SEQ ID NO: 268) | tcattccccttatgtc |
| | (SEQ ID NO: 267) | | (SEQ ID NO: 269) |

| | |
|---|---|
| Full VH | QVQLVQSGSELKKPGASVKVSCKASGYTFTTYGMNWVRQAPGQGLEWMGWINTNTGKPTYAQGFTGRFVFS |
| | LDASVSTAYLQISGLKADDTAVYYCARGYYGWDYHDYWGQGTLVTVSS |
| | (SEQ ID NO: 270) |
| DNA | caggtgcagctggtgcagtctgggtctgagttgaagaagcctggggcctcagtgaaggtttcctgcaaggc |
| | ttctggatacaccttcactacctatggtatgaattgggtgcgacaggcccctggacaagggcttgagtgga |
| | tgggatggatcaacaccaacactgggaagccaacgtatgcccagggcttcacaggacggtttgtcttctcc |
| | ttggacgcctctgtcagcacggcatatctgcagatcagcggcctaaaggctgacgacactgccgtgtatta |
| | ctgtgcgcgcggttactacggttgggactaccatgattactggggtcaaggtactctggtgaccgtctcct |
| | ca |
| | (SEQ ID NO: 271) |
| Full VL | SYVLTQPPSVSVAPGKTARITCGGNNIGSKSVHWYQQKPGQAPVLVIYYDSDRPSGIPERFSGSNSGNTAT |
| | LTISRVEAGDEADYYCQVWDSSSDHSPYVFGTGTKLTVLG |
| | (SEQ ID NO: 272) |
| DNA | tcctatgtgctgactcagccaccctcagtgtcagtggccccaggaaagacggccaggattacctgtggggg |
| | aaacaacattggaagtaaaagtgtgcactggtaccagcagaagccaggccaggcccctgtgctggtcatct |
| | attatgatagcgaccggccctcagggatccctgagcgattctctggctccaactctgggaacacggccacc |
| | ctgaccatcagcagggtcgaagccggggatgaggccgactattactgtcaggtgtgggatagtagtagtga |
| | tcattccccttatgtcttcggaactgggaccaagctgaccgtcctaggt |
| | (SEQ ID NO: 273) |
| scFv | SYVLTQPPSVSVAPGKTARITCGGNNIGSKSVHWYQQKPGQAPVLVIYYDSDRPSGIPERFSGSNSGNTAT |
| | LTISRVEAGDEADYYCQVWDSSSDHSPYVFGTGTKLTVLGSRGGGGSGGGGSGGGGSLEMAQVQLVQSGSE |
| | LKKPGASVKVSCKASGYTFTTYGMNWVRQAPGQGLEWMGWINTNTGKPTYAQGFTGRFVFSLDASVSTAYL |
| | QISGLKADDTAVYYCARGYYGWDYHD |
| | (SEQ ID NO: 274) |
| DNA | tcctatgtgctgactcagccaccctcagtgtcagtggccccaggaaagacggccaggattacctgtggggg |
| | aaacaacattggaagtaaaagtgtgcactggtaccagcagaagccaggccaggcccctgtgctggtcatct |
| | attatgatagcgaccggccctcagggatccctgagcgattctctggctccaactctgggaacacggccacc |
| | ctgaccatcagcagggtcgaagccggggatgaggccgactattactgtcaggtgtgggatagtagtagtga |
| | tcattccccttatgtcttcggaactgggaccaagctgaccgtcctaggttctagaggtggtggtggtagcg |
| | gcggcggcggctctggtggtggtggatccctcgagatggcccaggtgcagctggtgcagtctgggtctgag |
| | ttgaagaagcctggggcctcagtgaaggtttcctgcaaggcttctggatacaccttcactacctatggtat |
| | gaattgggtgcgacaggcccctggacaagggcttgagtggatgggatggatcaacaccaacactgggaagc |
| | caacgtatgcccagggcttcacaggacggtttgtcttctccttggacgcctctgtcagcacggcatatctg |
| | cagatcagcggcctaaaggctgacgacactgccgtgtattactgtgcgcgcggttactacggttgggacta |
| | ccatgattactggggtcaaggtactctggtgaccgtctcctca |
| | (SEQ ID NO: 275) |

TABLE 9

| Antigen | WT-1 (Ext002B #39) | | |
|---|---|---|---|
| Peptide | CMTWNQMNL (SEQ ID NO: 239) | | |
| CDRs: | 1 | 2 | 3 |
| VH | GGTFSSYAIS | GIIPIFGTANYAQKFQG | WFYMQAGDH |
| | (SEQ ID NO: 276) | (SEQ ID NO: 277) | (SEQ ID NO: 278) |
| DNA | ggaggcaccttcagcagctatg | gggatcatccctatctttggta | tggttctacatgcaggctggtg |
| | ctatcagc | cagcaaactacgcacagaagtt | atcat |
| | (SEQ ID NO: 279) | ccagggc | (SEQ ID NO: 281) |
| | | (SEQ ID NO: 280) | |
| VL | TGSSSDVGTYNYDS | DVSERPS | SSFAASSPWL |
| | (SEQ ID NO: 282) | (SEQ ID NO: 283) | (SEQ ID NO: 284) |
| DNA | actggaagcagcagtgatgttg | gatgtcagtgagcggccctca | agctcatttgcagccagcagtc |
| | gtacttataactatgactct | (SEQ ID NO: 286) | cctggctg |
| | (SEQ ID NO: 285) | | (SEQ ID NO: 287) |

| | |
|---|---|
| Full VH | QVQLVESGAEVKKPGSSVKVSCKASGGTFSSYAISWVRQAPGQGLEWMGGIIPIFGTANYAQKFQGRV |
| | TITADESTSTAYMELSSLRSEDTAVYYCARWFYMQAGDHWGQGTLVTVSS |
| | (SEQ ID NO: 288) |
| DNA | caggtgcagctggtggagtctggggctgaggtgaagaagcctgggtcctcggtgaaggtctcctgcaa |
| | ggcttctggaggcaccttcagcagctatgctatcagctgggtgcgacaggcccctggacaagggcttg |
| | agtggatgggagggatcatccctatctttggtacagcaaactacgcacagaagttccagggcagagtc |
| | acgattaccgcggacgaatccacgagcacagcctacatggagctgagcagcctgagatctgaggacac |
| | ggccgtgtattactgtgcgcgctggttctacatgcaggctggtgatcattggggtcaaggtactctgg |
| | tgaccgtctcctca |
| | (SEQ ID NO: 289) |
| Full VL | QAVLTQPASVSGSPGQSITISCTGSSSDVGTYNYDSWYQQHPGKAPKLMIYDVSERPSGVSNRFSGSK |
| | SGNTAFLTISGLQAEDEADYYCSSFAASSPWLFGGGTKVTVLG |
| | (SEQ ID NO: 290) |
| DNA | caggctgtgctgactcagcctgcctccgtgtctgggtctcctggacagtcgatcaccatctcctgcac |
| | tggaagcagcagtgatgttggtacttataactatgactcttggtaccaacagcacccaggcaaagccc |
| | ccaaactcatgatttatgatgtcagtgagcggccctcaggggtttctaatcgcttctccggctccaag |
| | tctggcaacacggccttcctgaccatctctgggctccaggctgaggacgaggctgattattactgcag |
| | ctcatttgcagccagcagtccctggctgttcggcggagggaccaaggtcaccgtcctaggt |
| | (SEQ ID NO: 291) |
| scFv | QAVLTQPASVSGSPGQSITISCTGSSSDVGTYNYDSWYQQHPGKAPKLMIYDVSERPSGVSNRFSGSK |
| | SGNTAFLTISGLQAEDEADYYCSSFAASSPWLFGGGTKVTVLGSRGGGGSGGGGSGGGGSLEMAQVQL |
| | VESGAEVKKPGSSVKVSCKASGGTFSSYAISWVRQAPGQGLEWMGGIIPIFGTANYAQKFQGRVTITA |
| | DESTSTAYMELSSLRSEDTAVYYCARWFYMQAGDHWGQGTLVTVSS |
| | (SEQ ID NO: 292) |
| DNA | caggctgtgctgactcagcctgcctccgtgtctgggtctcctggacagtcgatcaccatctcctgcac |
| | tggaagcagcagtgatgttggtacttataactatgactcttggtaccaacagcacccaggcaaagccc |
| | ccaaactcatgatttatgatgtcagtgagcggccctcaggggtttctaatcgcttctccggctccaag |
| | tctggcaacacggccttcctgaccatctctgggctccaggctgaggacgaggctgattattactgcag |
| | ctcatttgcagccagcagtccctggctgttcggcggagggaccaaggtcaccgtcctaggttctagag |
| | gtggtggtggtagcggcggcggcggctctggtggtggtggatccctcgagatggcccaggtgcagctg |
| | gtggagtctggggctgaggtgaagaagcctgggtcctcggtgaaggtctcctgcaaggcttctggagg |
| | caccttcagcagctatgctatcagctgggtgcgacaggcccctggacaagggcttgagtggatgggag |
| | ggatcatccctatctttggtacagcaaactacgcacagaagttccagggcagagtcacgattaccgcg |
| | gacgaatccacgagcacagcctacatggagctgagcagcctgagatctgaggacacggccgtgtatta |
| | ctgtgcgcgctggttctacatgcaggctggtgatcattggggtcaaggtactctggtgaccgtctcct |
| | ca |
| | (SEQ ID NO: 293) |

TABLE 10

| Antigen | WT-1 (Ext002B #40) | | |
|---|---|---|---|
| Peptide | CMTWNQMNL (SEQ ID NO: 239) | | |
| CDRs: | 1 | 2 | 3 |
| VH | GFTFSSYGMN | SISSSSSYIYYADSVKG | IQDATGEEMILYDY |
| | (SEQ ID NO: 294) | (SEQ ID NO: 295) | (SEQ ID NO: 296) |
| DNA | ggattcaccttcagtagctatggc | tccattagtagtagtagtagttac | atccaggacgctactggtgaa |
| | atgaac | atatactacgcagactcagtgaag | gaaatgatcctgtacgattac |
| | (SEQ ID NO: 297) | ggc | (SEQ ID NO: 299) |
| | | (SEQ ID NO: 298) | |
| VL | RSSQSLVYSDGNTYLN | QVSKRDS | MQGSHLRT |
| | (SEQ ID NO: 300) | (SEQ ID NO: 301) | (SEQ ID NO: 302) |
| DNA | aggtctagtcaaagcctcgtatac | caggtttctaagcgggactct | atgcaaggttcacacttgcgg |
| | agtgatggaaacacctatttgaat | (SEQ ID NO: 304) | acg |
| | (SEQ ID NO: 303) | | (SEQ ID NO: 305) |

| | |
|---|---|
| Full VH | QVQLVESGGGLVKPGGSLRLSCAASGFTFSSYGMNWVRQAPGKGLEWVSSISSSSSYIYYADSVKGRFTIS |
| | RDNAKNSLYLQMNSLRAEDTAVYYCARIQDATGEEMILYDYWGQGTLVTVSS |
| | (SEQ ID NO: 306) |
| DNA | caggtgcagctggtggagtctgggggaggcctggtcaagcctggggggtccctgaggctctcctgtgcagc |
| | ctctggattcaccttcagtagctatggcatgaactgggtccgccaggctccagggaaggggctggagtggg |
| | tctcatccattagtagtagtagtagttacatatactacgcagactcagtgaagggccgattcaccatctcc |
| | agagacaacgccaagaactcactgtatctgcaaatgaacagcctgagagccgaggacacggctgtgtatta |
| | ctgtgcgcgcatccaggacgctactggtgaagaaatgatcctgtacgattactggggtcaaggtactctgg |
| | tgaccgtctcctca |
| | (SEQ ID NO: 307) |
| Full VL | EIVLTQSPLSLPVTLGQPASISCRSSQSLVYSDGNTYLNWFQQRPGQSPRRLIYQVSKRDSGVPDRFSGSG |
| | SGTDFTLKISRVEAEDVGMYYCMQGSHLRTFGQGTKVEIKR |
| | (SEQ ID NO: 308) |
| DNA | attgtgctgactcagtctccactctccctgcccgtcacccttggacagccggcctccatctcctgcaggtc |
| | tagtcaaagcctcgtatacagtgatggaaacacctatttgaattggtttcagcagaggccaggccaatctc |
| | caaggcgcctaatttatcaggtttctaagcgggactctggggtcccagacagattcagcggcagtgggtca |
| | ggcactgatttcacactgaaaatcagcagggtggaggctgaggatgttgggatgtattactgcatgcaagg |
| | ttcacacttgcggacgttcggccaagggaccaaggtggaaatcaaacgt |
| | (SEQ ID NO: 309) |
| scFv | EIVLTQSPLSLPVTLGQPASISCRSSQSLVYSDGNTYLNWFQQRPGQSPRRLIYQVSKRDSGVPDRFSGSG |
| | SGTDFTLKISRVEAEDVGMYYCMQGSHLRTFGQGTKVEIKRSRGGGGSGGGGSGGGGSLEMAQVQLVESGG |
| | GLVKPGGSLRLSCAASGFTFSSYGMNWVRQAPGKGLEWVSSISSSSSYIYYADSVKGRFTISRDNAKNSLY |
| | LQMNSLRAEDTAVYYCARIQDATGEEMILYDYWGQGTLVTVSS |
| | (SEQ ID NO: 310) |
| DNA | gaaattgtgctgactcagtctccactctccctgcccgtcacccttggacagccggcctccatctcctgcag |
| | gtctagtcaaagcctcgtatacagtgatggaaacacctatttgaattggtttcagcagaggccaggccaat |
| | ctccaaggcgcctaatttatcaggtttctaagcgggactctggggtcccagacagattcagcggcagtggg |
| | tcaggcactgatttcacactgaaaatcagcagggtggaggctgaggatgttgggatgtattactgcatgca |
| | aggttcacacttgcggacgttcggccaagggaccaaggtggaaatcaaacgttctagaggtggtggtggta |
| | gcggcggcggcggctctggtggtggtggatccctcgagatggcccaggtgcagctggtggagtctggggga |
| | ggcctggtcaagcctggggggtccctgaggctctcctgtgcagcctctggattcaccttcagtagctatgg |
| | catgaactgggtccgccaggctccagggaaggggctggagtgggtctcatccattagtagtagtagtagtt |
| | acatatactacgcagactcagtgaagggccgattcaccatctccagagacaacgccaagaactcactgtat |
| | ctgcaaatgaacagcctgagagccgaggacacggctgtgtattactgtgcgcgcatccaggacgctactgg |
| | tgaagaaatgatcctgtacgattactggggtcaaggtactctggtgaccgtctcctca |
| | (SEQ ID NO: 311) |

TABLE 11

| Antigen | WT-1 (Ext002B #41) | | |
|---|---|---|---|
| Peptide | CMTWNQMNL (SEQ ID NO: 239) | | |
| CDRs: | 1 | 2 | 3 |
| VH | GYTFTDYYIH | WMNPNSGNSVSAQKFQG | YQGSTWKYDSYGDL |
| | (SEQ ID NO: 312) | (SEQ ID NO: 313) | (SEQ ID NO: 314) |
| DNA | ggatacaccttcaccg | tggatgaaccctaacagtgggaactc | taccagggttctacttggaaat |
| | actactatatacac | agtctctgcacagaagttccagggc | acgactcttacggtgatctg |
| | (SEQ ID NO: 315) | (SEQ ID NO: 316) | (SEQ ID NO: 317) |
| VL | GGNEIGFNGVH | NNRVRPS | QVWVNPDNEYV |
| | (SEQ ID NO: 318) | (SEQ ID NO: 319) | (SEQ ID NO: 320) |
| DNA | gggggaaacgagattggatttaa | aacaatagggtccggccctca | caggtgtgggttaatcctgata |
| | tggtgttcat | (SEQ ID NO: 322) | atgaatatgtc |
| | (SEQ ID NO: 321) | | (SEQ ID NO: 323) |

| | |
|---|---|
| Full VH | QVQLVQSGAEVKKPGASVKVSCKASGYTFTDYYIHWVRQAPGQGLEWMGWMNPNSGNSVSAQKFQGRVTMTRD |
| | TSINTAYMELSSLTSDDTAVYYCARYQGSTWKYDSYGDLWGQGTLVTVTS |
| | (SEQ ID NO: 324) |
| DNA | caggtccagctggtgcagtctggggctgaggtgaagaagcctggggcctcagtgaaggtctcctgcaaggctt |
| | ctggatacaccttcaccgactactatatacactgggtgcggcaggcccctggacaagggctggagtggatggg |
| | atggatgaaccctaacagtgggaactcagtctctgcacagaagttccagggcagagtcaccatgaccagggat |
| | acctccataaacacagcctacatggagctgagcagcctgacatctgacgacacggccgtatattactgtgcgc |
| | gctaccagggttctacttggaaatacgactcttacggtgatctgtggggtcaaggtactctggtgaccgtcac |
| | ctca |
| | (SEQ ID NO: 325) |
| Full VL | QAVLTQPPSVSVAPGETATVTCGGNEIGFNGVHWYKQKAGQAPLLVIYNNRVRPSGISERLSGSNSGNTATLT |
| | ISRVEAGDEADYYCQVWVNPDNEYVFGSGTKVTVLG |
| | (SEQ ID NO: 326) |
| DNA | caggctgtgctgactcagccaccctcggtgtcagtggccccaggagagacggccactgttacctgtgggggaa |
| | acgagattggatttaatggtgttcattggtataagcagaaggcaggccaggcccctctgttggtcatctataa |
| | caatagggtccggccctcagggatctctgagcgactctctggctccaactctggtaacacggccaccctgacc |
| | atcagcagggtcgaagccggggatgaggccgactattactgtcaggtgtgggttaatcctgataatgaatatg |
| | tcttcggatcggggaccaaggtcaccgtcctaggt |
| | (SEQ ID NO: 327) |
| scFv | QAVLTQPPSVSVAPGETATVTCGGNEIGFNGVHWYKQKAGQAPLLVIYNNRVRPSGISERLSGSNSGNTATLT |
| | ISRVEAGDEADYYCQVWVNPDNEYVFGSGTKVTVLGSRGGGGSGGGGSGGGGSLEMAQVQLVQSGAEVKKPGA |
| | SVKVSCKASGYTFTDYYIHWVRQAPGQGLEWMGWMNPNSGNSVSAQKFQGRVTMTRDTSINTAYMELSSLTSD |
| | DTAVYYCARYQGSTWKYDSYGDLWGQGTLVTVTS |
| | (SEQ ID NO: 328) |
| DNA | caggctgtgctgactcagccaccctcggtgtcagtggccccaggagagacggccactgttacctgtgggggaa |
| | acgagattggatttaatggtgttcattggtataagcagaaggcaggccaggcccctctgttggtcatctataa |
| | caatagggtccggccctcagggatctctgagcgactctctggctccaactctggtaacacggccaccctgacc |
| | atcagcagggtcgaagccggggatgaggccgactattactgtcaggtgtgggttaatcctgataatgaatatg |
| | tcttcggatcggggaccaaggtcaccgtcctaggttctagaggtggtggtggtagcggcggcggcggctctgg |
| | tggtggtggatccctcgagatggcccaggtccagctggtgcagtctggggctgaggtgaagaagcctggggcc |
| | tcagtgaaggtctcctgcaaggcttctggatacaccttcaccgactactatatacactgggtgcggcaggccc |
| | ctggacaagggctggagtggatgggatggatgaaccctaacagtgggaactcagtctctgcacagaagttcca |
| | gggcagagtcaccatgaccagggatacctccataaacacagcctacatggagctgagcagcctgacatctgac |
| | gacacggccgtatattactgtgcgcgctaccagggttctacttggaaatacgactcttacggtgatctgtggg |
| | gtcaaggtactctggtgaccgtcacctca |
| | (SEQ ID NO: 329) |

TABLE 12

| Antigen | WT-1 (Ext002B #43) | | |
|---|---|---|---|
| Peptide | CMTWNQMNL (SEQ ID NO: 239) | | |
| CDRs: | 1 | 2 | 3 |
| VH | GFTFSSYEMN | YISSSGSTIYYADSVKG | DWRSSYYYSQYDK |
| | (SEQ ID NO: 330) | (SEQ ID NO: 331) | (SEQ ID NO: 332) |
| DNA | ggattcaccttcagtagttatga | tacattagtagtagtggtagtac | gactggcgttcttcttactacta |
| | aatgaac | catatactacgcagactctgtga | ctctcagtacgataaa |
| | (SEQ ID NO: 333) | agggc | (SEQ ID NO: 335) |
| | | (SEQ ID NO: 334) | |
| VL | TRSSGNIASNYVQ | ADNQRPS | QSYENNIHV |
| | (SEQ ID NO: 336) | (SEQ ID NO: 337) | (SEQ ID NO: 338) |
| DNA | acccgcagcagtggcaacattgc | gcggacaaccaaagaccctct | cagtcttatgaaaacaacattca |
| | cagcaactatgtgcag | (SEQ ID NO: 340) | cgtg |
| | (SEQ ID NO: 339) | | (SEQ ID NO: 341) |

| | |
|---|---|
| Full VH | EVQLVESGGGLVQPGESLRLSCAASGFTFSSYEMNWVRQAPGKGLEWVSYISSSGSTIYYADSVKGRFTISR |
| | DNAKNSLYLQMNSLRAEDTAVYYCARDWRSSYYYSQYDKWGQGTLVTVSS |
| | (SEQ ID NO: 342) |
| DNA | gaggtgcagctggtggagtctgggggaggcttggtacagcctggagagtccctgagactctcctgtgcagcc |
| | tctggattcaccttcagtagttatgaaatgaactgggttcgccaggctccagggaaggggctggagtgggtt |
| | tcatacattagtagtagtggtagtaccatatactacgcagactctgtgaagggccgattcaccatctccaga |
| | gacaacgccaagaactcactgtatctgcaaatgaacagcctgagagccgaggacacggctgtgtattactgt |
| | gcgcgcgactggcgttcttcttactactactctcagtacgataaatggggtcaaggtactctggtgaccgtc |
| | tcctca |
| | (SEQ ID NO: 343) |
| Full VL | NFMLTQPHSVSESPGKTVSISCTRSSGNIASNYVQWYQHRPGRSPTTVIYADNQRPSGVPDRFSGSIDTSSN |
| | SASLTISGLRTEDEADYYCQSYENNIHVFGGGTKLTVLG |
| | (SEQ ID NO: 344) |
| DNA | aattttatgctgactcagccccactctgtgtcggagtctccggggaagacggtaagcatctcctgcacccgc |
| | agcagtggcaacattgccagcaactatgtgcagtggtaccaacaccgcccgggccgttcccccaccactgtg |
| | atctatgcggacaaccaaagaccctctggggtccctgatcgcttctctggctccatcgacacctcctccaac |
| | tctgcctccctcaccatctctggactgaggactgaggacgaggctgactactactgtcagtcttatgaaaac |
| | aacattcacgtgttcggcggggggaccaagctgaccgtcctaggt |
| | (SEQ ID NO: 345) |
| scFv | NFMLTQPHSVSESPGKTVSISCTRSSGNIASNYVQWYQHRPGRSPTTVIYADNQRPSGVPDRFSGSIDTSSN |
| | SASLTISGLRTEDEADYYCQSYENNIHVFGGGTKLTVLGSRGGGGSGGGGSGGGGSLEMAEVQLVESGGGLV |
| | QPGESLRLSCAASGFTFSSYEMNWVRQAPGKGLEWVSYISSSGSTIYYADSVKGRFTISRDNAKNSLYLQMN |
| | SLRAEDTAVYYCARDWRSSYYYSQYDKWGQGTLVTVSS |
| | (SEQ ID NO: 346) |
| DNA | aattttatgctgactcagccccactctgtgtcggagtctccggggaagacggtaagcatctcctgcacccgc |
| | agcagtggcaacattgccagcaactatgtgcagtggtaccaacaccgcccgggccgttcccccaccactgtg |
| | atctatgcggacaaccaaagaccctctggggtccctgatcgcttctctggctccatcgacacctcctccaac |
| | tctgcctccctcaccatctctggactgaggactgaggacgaggctgactactactgtcagtcttatgaaaac |
| | aacattcacgtgttcggcggggggaccaagctgaccgtcctaggttctagaggtggtggtggtagcggcggc |
| | ggcggctctggtggtggtggatccctcgagatggccgaggtgcagctggtggagtctgggggaggcttggta |
| | cagcctggagagtccctgagactctcctgtgcagcctctggattcaccttcagtagttatgaaatgaactgg |
| | gttcgccaggctccagggaaggggctggagtgggtttcatacattagtagtagtggtagtaccatatactac |
| | gcagactctgtgaagggccgattcaccatctccagagacaacgccaagaactcactgtatctgcaaatgaac |
| | agcctgagagccgaggacacggctgtgtattactgtgcgcgcgactggcgttcttcttactactactctcag |
| | tacgataaatggggtcaaggtactctggtgaccgtctcctca |
| | (SEQ ID NO: 347) |

In the sequences in Tables 1-14, bolded text indicates a linker sequence between hypervariable heavy and light chain sequences.

In some embodiments, anti-WT-1 antibodies used in the method of the invention may further encompass those comprising light and heavy hypervariable regions and constant regions, for example as shown in Tables 13 (heavy chain), 14 (light chain) and 15 (constant regions). Similarly, the CDRs of other WT-1 antibodies suitable for use in practicing the disclosed method are shown in FIGS. 7-10.

TABLE 13

| | CDR-H1 | CDR-H2 | CDR-H3 | SEQ ID NO. |
|---|---|---|---|---|
| Group I | | | | |
| EXT002-12(166) | SNAVAWN | RTYRGSTYY---ALSV | G-SNSAFDF | 119-121 |
| EXT002-5(184) | SNSAAWN | RTYYGSKWYNDYAVSV | GRLGDAFDI | 122-124 |
| EXT002-8(184) | SDGAAWN | RTYYRSKWYNDYAVSV | GDYYYGMDV | 125-127 |
| Consensus(191) | SNAAAWN | RTYYGSKWYNDYAVSV | G AFDI | 128-130 |
| Group II | | | | |
| EXT002-14(163) | SYWIS | RIDPSDSYTNYSPSFQG | GD------YDFYLDP-- | 131-133 |
| EXT002-25(163) | SYGIS | WISAYNGNTNYAQKLQG | DLYSSGWYESYYYGMDV | 134-136 |
| EXT002-3(186) | SYAIS | GIIPIFGTANYAQKFQG | RIP-P------YYGMDV | 137-139 |
| EXT002-30(163) | SYGIS | WISAHNGNTNYAQKLQG | DR-------VWFGDLSD | 134, 140, 141 |
| EXT002-33(163) | SYAIS | GIIPIFGTANYAQKEQG | NYDFWSG-----DAFDI | 137, 142, 143 |
| Consensus(188) | SYAIS | I P G TNYAQKFQG | FY GMDV | 137, 144, 145 |
| Group III | | | | |
| EXT002-34(161) | DYGMS | GINWNGGSTGYADSV | ERGY-GYHDPHDY | 146-148 |
| EXT002-40(157) | NYTMN | SISLSGAYIYYADSL | EGYSSSVYDAFDL | 149-151 |
| EXT002-45(165) | SYGMH | GILSDGGKDYYVDSV | CSSN-YGNDAFDI | 152-154 |
| EXT002-48(165) | TYSMN | SISSGAYSIFYADSV | DQYYGDKWDAFDI | 155-157 |
| Consensus(170) | SYGMN | SISSGGSIYYADSV | E YY WDAFDI | 158-160 |

TABLE 14

| | | | | SEQ |
|---|---|---|---|---|
| | CDR-L1 | CDR-L2 | CDR-L3 | ID NOS. |
| Group I | | | | |
| EXT002-1 (46) | CSGSSSNIGS-NTVN | SNNQRPSG | AAWDDSLNG--WVFG | 161-163 |
| EXT002-10 (46) | CSGSSSNIGS-NTVN | SNNQRPSG | EAWDDSLKG--PVFG | 161, 162, 164 |
| EXT002-12 (22) | CTGSSSNIGAGYDVH | GNSNRPSG | QSYDSSLSADNYVFG | 165-167 |
| EXT002-13 (46) | CSGSSSNIGS-NTVN | SNNQRPSG | AAWDDSLNG--WVFG | 161-163 |
| EXT002-2 (46) | CSGSSSNIGR-NIVN | SNIERPSG | ASWDDSLNG--VLFG | 168-170 |
| EXT002-20 (46) | CSGSRSNIAS-NGVG | KNDQRPSG | SAWDDSLDGH-VVFG | 171-173 |
| EXT002-23 (46) | CTGSSSNIGAGYDVH | GNSNRPSG | AAWDDSLNG--YVFG | 165, 166, 174 |
| EXT002-25 (22) | CSGSSSNIGS-STVN | SNSQRPSG | AAWDDSLNG--VVFG | 175-177 |
| EXT002-3 (46) | CSGSSSNIGS-NYVY | RSNQRPSG | AAWDDSLNG--VVFG | 178, 179, 177 |
| EXT002-30 (22) | CSGSSSNIGR-NTVN | SNNQRPSG | AAWDDSLNG--YVFG | 180, 162, 174 |
| EXT002-33 (22) | CSGSSSNIGN-DYVS | DNNKRPSG | GTWDNSLSA--WVFG | 181-183 |
| EXT002-36 (22) | CSGSSSNIGS-NSVY | NNNQRPSG | ATWDDSLSG--WVFG | 184-186 |
| EXT002-40 (22) | CSGSSSNIGS-NYVY | RNNQRPSG | AAWDDSLSA--WVFG | 178, 187, 188 |
| EXT002-42 (46) | CSGSTSNIGS-YYVS | DNNNRPSG | GTWDSSLSA--WVFG | 189-191 |
| EXT002-45 (22) | CSGSSSNIGN-NYVS | DNNKRPSG | GTWDSSLSA--WVFG | 192, 182, 191 |
| EXT002-48 (22) | CSGSNSNIGT-NTVT | SNFERPSG | SAWDDSFNG--PVFG | 193-195 |
| EXT002-6 (46) | CSGSSSNIGS-NYVS | RNNQRPSG | AAWDDGLRG--YVFG | 196, 187, 197 |
| EXT002-9 (22) | CSGSSSNIGS-NTVN | SNNQRPSG | EAWDDSLKG--PVFG | 161, 162, 164 |
| Consensus (46) | CSGSSSNIGS N V | NNQRPSG | AAWDDSL G WVFG | 161-163 |
| Group II | | | | |
| EXT002-24 (24) | RASQSISSYLN | AASSLQS | QQSYSTP--T | 198-200 |
| EXT002-31 (24) | RASQGISNYLA | AASTLQS | QKYNSAPGVT | 201-203 |
| EXT002-35 (24) | RASQSINGWLA | RASTLQS | QQSSSLP-FT | 204-206 |
| EXT002-5 (48) | RASQSISSYLN | AASSLQS | QQSYSTP-LT | 198-200 |
| EXT002-7 (48) | RASQGISYYLA | AASTLKS | QQLNSYP-LT | 207-209 |
| EXT002-B (48) | RASQSISSYLN | AASSLQS | QQSYSTP-WT | 198-200 |
| Consensus (48) | RASQSISSYLN | AASSLQS | QQSYSTP LT | 198-200 |
| Group III | | | | |
| EXT002-16 (23) | GGNNIGSKSVH | DDSDRPS | QVWDSSSDHPV | 210-212 |
| EXT002-17 (47) | GGNNIGSKSVH | DDSDRPS | QVWDSSGDHPV | 210, 211, 213 |
| EXT002-19 (47) | GGNNIGSKSVH | YDSDRPS | QVWDSSSDHPV | 210, 214, 212 |
| EXT002-21 (19) | GGTNIGSRFVH | DDSDRPS | QVWDSSGDHPV | 215, 211, 213 |
| EXT002-22 (47) | GGNNVESKSVH | YDRDRPS | EVWDSGSDHPV | 216-218 |
| EXT002-32 (23) | GGKNIGSKSVH | YDSDRPS | QVWDSGSDHYV | 219, 214, 220 |
| EXT002-34 (23) | GGNNIGSKSVH | DDSDRPS | QVWISSGDRVI | 210, 211, 221 |
| EXT002-43 (23) | GGDNIGSQGVH | YDTDRPS | QVWGASSDHPV | 222-224 |
| Consensus (47) | GGNNIGSKSVH | YDSDRPS | QVWDSSSDHPV | 210, 214, 212 |
| Group IV | | | | |
| EXT002-11 (47) | TGTSSDVGGYNYVS | DVSKRPS | GIYTYSDSW--V | 225-227 |
| EXT002-14 (23) | TGTSSDVGGYNYVS | DVGNRPS | SSYTSSSTR--V | 225, 228, 229 |
| EXT002-26 (23) | TGTRSDVGLYNYVA | DVIYRPG | SSYTNTGTV--L | 230-232 |
| EXT002-4 (47) | TGTSSDFGDYDYVS | DVSDRPS | QSYDSSLSGSGV | 233-235 |
| Consensus (47) | TGTSSDVGGYNYVS | DVS RPS | SSYTSS S V | 225, 234, 229 |

TABLE 15
Constant Regions

| | |
|---|---|
| Human heavy chain | ASTKGPSVFPLAPSSKSTSGGTAALGCLVKDYFPEPVTVSWNSGAL |
| constant region and | TSGVHTFPAVLQSSGLYSLSSVVTVPSSSLGTQTYICNVNHKPSNT |
| IgG1 Fc domain | KVDKRVEPKSCDKTHTCPPCPAPELLGGPSVFLFPPKPKDTLMISR |
| sequence | TPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQ |
| | VYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQK |
| | SLSLSPGK |
| | (SEQ ID NO. 236) |
| Human light chain | TVAAPSVFIFPPSDEQLKSGTASVVCLLNNFYPREAKVQWKVDNAL |
| (kappa) | QSGNSQESVTEQDSKDSTYSLSSTLTLSKADYEKHKVYACEVTHQG |
| | LSSPVTKSFNRGEC |
| | (SEQ ID NO. 237) |
| Human light chain | QPKANPTVTLFPPSSEELQANKATLVCLISDFYPGAVTVAWKADGS |
| (lambda) | PVKAGVETTKPSKQSNNKYAASSYLSLTPEQWKSHRSYSCQVTHEG |
| | STVEKTVAPTECS |
| | (SEQ ID NO. 238) |

In some embodiments, the anti-WT-1 antibodies are those in which the constant region/framework region is altered, for example, by amino acid substitution, to modify the properties of the antibody (e.g., to increase or decrease one or more of: antigen binding affinity, Fc receptor binding, antibody carbohydrate, for example, glycosylation, fucosylation etc, the number of cysteine residues, effector cell function, effector cell function, complement function or introduction of a conjugation site).

In one embodiment, the antibody is an anti-WT-1/A2 antibody and comprises the human IgG1 constant region and Fc domain shown in Table 9. In one embodiment, the anti-WT-1/A2 antibody comprises a human kappa sequence, or a human lambda sequence having the sequence set forth in Table 9. The amino acid sequences for some complementarity determining regions (CDRs) of antibodies of the invention are shown in Tables 1-14 and in FIGS. 7-10.

Glycosylation (specifically fucosylation) variants of IgG Fc can be produced using host expression cells and methods described in U.S. Pat. Nos. 8,025,879; 8,080,415; and 8,084,022, the contents of which are incorporated by reference. Briefly, messenger RNA (mRNA) coding for heavy or light chain of the antibodies disclosed herein, is obtained by employing standard techniques of RNA isolation purification and optionally size based isolation. cDNAs corresponding to mRNAs coding for heavy or light chain are then produced and isolated using techniques known in the art, such as cDNA library construction, phage library construction and screening or RT-PCR using specific relevant primers. In some embodiments, the cDNA sequence may be one that is wholly or partially manufactured using known in vitro DNA manipulation techniques to produce a specific desired cDNA. The cDNA sequence can then be positioned in a vector which contains a promoter in reading frame with the gene and compatible with the low fucose-modified host cell.

According to an aspect of some embodiments of the disclosure there is provided a method of treating cancer in a subject in need thereof. The method, according to these embodiments, is effected by administering to the subject a therapeutically effective amount of a tyrosine kinase inhibitor and a therapeutically effective amount of an anti-WT-1 antibody.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition, and includes, for example, reducing a size of a tumor in a subject, effecting a state of remission in a subject, increasing an expected survival probability, increasing life expectancy, and increasing an expected time to disease progression.

As described in the Examples section that follows, tyrosine kinase inhibitors such as imatinib and dasatinib and anti-WT-1 antibodies were surprisingly observed to have a beneficial additive effect when administered together. Importantly, several animals administered the combination of dasatinib and anti-WT-1 antibody appeared to be cured of their disease whereas animals administered either drug alone were not.

Suitable routes of administration for the TKI may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer the pharmaceutical composition in a local rather than systemic manner, for example, via injection of the pharmaceutical composition directly into a tissue region of a patient.

Oral administration is an exemplary administration for tyrosine kinase inhibitors. It is to be understood that administration of a tyrosine kinase inhibitor and anti-WT-1 antibody need not be via the same route, and need not be performed simultaneously.

WT-1 (or anti-WT-1) antibodies will vary in the nature of the antigen to which they bind. Specificity is determined by HLA antigen type. For example, HLA-A*0201 is expressed in 39-46% of all Caucasians and therefore, an antibody with specificity for WT-1 peptide in conjunction with HLA-A2 represents a suitable choice of antibody for use in the Caucasian population. Anti-WT-1 antibodies with specificity for a WT-1 peptide presented on the surface of cancer cells in conjunction with HLA-A24 may be more appropriate for use in New World natives and Asian populations in which the HLA-A24 target is particularly expressed. Choice of WT-1 antibody, therefore, may depend on HLA type of the subject to whom it is to be administered.

In other embodiments, the anti-WT-1/HLA antibodies may comprise one or more framework region amino acid substitutions designed to improve protein stability, antibody binding, expression levels or to introduce a site for conjugation of therapeutic agents. These scFv are then used to produce recombinant human monoclonal Igs in accordance with methods known to those of skill in the art.

Methods for reducing the proliferation of leukemia cells is also included, comprising contacting leukemia cells with a WT-1 antibody of the invention. In a related aspect, the antibodies of the invention can be used for the prevention or treatment of leukemia. Administration of therapeutic antibodies is known in the art.

### Pharmaceutical Compositions and Methods of Treatment

WT-1 antibodies can be administered for therapeutic treatments to a patient suffering from a tumor or WT-1-associated pathologic condition in an amount sufficient to prevent, inhibit, or reduce the progression of the tumor or pathologic condition. Progression includes, e.g, the growth, invasiveness, metastases and/or recurrence of the tumor or pathologic condition. Amounts effective for this use will depend upon the severity of the disease and the general state of the patient's own immune system. Dosing schedules will also vary with the disease state and status of the patient, and will typically range from a single bolus dosage or continuous infusion to multiple administrations per day (e.g., every 4-6 hours), or as indicated by the treating physician and the patient's condition.

The identification of medical conditions treatable by WT-1 antibodies of the present invention is well within the ability and knowledge of one skilled in the art. For example, human individuals who are either suffering from a clinically significant leukemic disease or who are at risk of developing clinically significant symptoms are suitable for administration of the present WT-1 antibodies. A clinician skilled in the art can readily determine, for example, by the use of clinical tests, physical examination and medical/family history, if an individual is a candidate for such treatment.

Non-limiting examples of pathological conditions characterized by WT-1 expression include chronic myelocytic leukemia, acute lymphoblastic leukemia (ALL), acute myeloid/myelogenous leukemia (AML) and myelodysplastic syndrome (MDS). Additionally, solid tumors, in general and in particular, tumors associated with mesothelioma, ovarian cancer, gastrointestinal cancers, breast cancer, prostate cancer and glioblastoma are amenable to treatment using WT-1 antibodies.

Any suitable method or route can be used to administer a WT-1 antibody of the present invention, and optionally, to coadminister antineoplastic agents and/or antagonists of other receptors. Routes of administration include, for example, oral, intravenous, intraperitoneal, subcutaneous, or intramuscular administration. It should be emphasized, however, that the present invention is not limited to any particular method or route of administration.

It is understood that WT-1 antibodies of the invention will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Other aspects of the invention include without limitation, the use of antibodies and nucleic acids that encode them for treatment of WT-1 associated disease, for diagnostic and prognostic applications as well as use as research tools for the detection of WT-1 in cells and tissues. Pharmaceutical compositions comprising the disclosed antibodies and nucleic acids are encompassed by the invention. Vectors comprising the nucleic acids of the invention for antibody-based treatment by vectored immunotherapy are also contemplated by the present invention. Vectors include expression vectors which enable the expression and secretion of antibodies, as well as vectors which are directed to cell surface expression of the antigen binding proteins, such as chimeric antigen receptors.

The method of the present invention will now be described in more detail with respect to representative embodiments.

### Example 1

### Materials and Methods

Cell Samples, Cell Lines and Antibodies.

After informed consent on Memorial Sloan-Kettering Cancer Center Institutional Review Board approved protocols, peripheral blood mononuclear cells (PBMC) from HLA-typed healthy donors and patients were obtained by Ficoll density centrifugation. All cells were HLA typed by the Department of Cellular Immunology at Memorial Sloan-Kettering Cancer Center. Leukemia cell line, BV173, (WT-1+, A0201+) was kindly provided by Dr. H. J. Stauss (University College London, London, United Kingdom). The cell lines were cultured in RPMI 1640 supplemented with 5% FCS, penicillin, streptomycin, 2 mmol/L glutamine, and 2-mercaptoethanol at 37° C./5% CO₂.

Animals.

Six to eight week-old male NOD.Cg-Prkdc scid IL2rgtm1WjI/SzJ mice, known as NOD/SCID gamma (NSG), were purchased from the Jackson Laboratory (Bar Harbor, Me.) or obtained from MSKCC animal breeding facility.

Transduction and Selection of Luciferase/GFP Positive Cells.

BV173 cells were engineered to express high level of GFP-luciferase fusion protein, using lentiviral vectors containing a plasmid encoding the luc/GFP (39). Using single cell cloning, only the cells showing high level GFP expression were selected by flow cytometry analysis and were maintained and used for the animal study.

### Example 2

### Antibody-Dependent Cellular Cytotoxicity (ADCC)

ADCC is considered to be one of the major effector mechanisms of therapeutic mAb in humans. Evaluation of efficacy, therefore, begins with in vitro experiments measuring ADCC against BV173 cell line, derived from CML in blastic crisis. Fresh BV173 cells were used for ADCC target cells. WT-1 antibody or its isotype control human IgG1 was incubated at 750 ng/ml with target cells and fresh PBMCs at different effector:target (E:T) ratio for 6 hrs. Imatinib was added at concentrations of 0, 1, 5, and 10 μM. The supernatants were harvested and the cytotoxicity was measured by standard chromium 51 release assay.

In the presence of human PBMC, WT-1 antibody mediated dose-dependent PBMC ADCC against naturally presented RMF epitope by HLA-A0201 molecule on tumor cells, the leukemia cell line BV173. Importantly, WT-1 antibody was able to mediate ADCC in the presence of various doses of imatinib. The killing was consistently observed at 750 ng/ml of WT-1 antibody using PBMCs as effector cells from multiple healthy donors. These results demonstrated that imatinib does not affect the ability of WT-1 antibody to mediate specific ADCC against cells that naturally express RMF and HLA-A0201 complex in vitro (FIG. 1).

### Example 3

In vivo efficacy of ESKM with TKIs was evaluated using NSG mice injected with HLA-A0201⁺ leukemic cell line BV173. The protocol used for imatinib and dasatinib therapy in combination with ESKM consisted of injecting 3×10⁶ cells per mouse via tail vein, luciferin imaging 6 days after injection to assess tumor engraftment, and initiation of therapy immediately after imaging on day 6. Luciferin imaging was used weekly to monitor tumor growth. The TKI is injected intraperitoneally daily (50 mg/kg for imatinib and 20-40 mg/kg for dasatinib.) The antibody is injected intravenously twice per week.

### Example 4

### Therapeutic Effects of Imatinib Plus Anti-WT-1/HLA Antibody (ESKM) in a Human Leukemia Xenograft NSG Model

Three million BV173 human leukemia cells were injected IV by tail vein into NSG mice. On day 6, tumor engraftment was confirmed by firefly luciferase imaging in all mice that were to be treated; mice were then randomly divided into different treatment groups (A, B, C, and D). Immediately after imaging on day 6, therapy was initiated with anti-WT-1 antibody ESKM 100 μg administered by intraperitoneal (IP) injection twice weekly. Imatinib was also administered by IP injection at 50 mg/kg daily. Therapy continued for 5 weeks (10 doses of ESKM and 34 doses of imatinib per mouse). Group A: No therapy; Group B: imatinib treatment only; Group C: ESK treatment only; Group D: combination of both imatinib daily and ESK twice weekly. Tumor growth was assessed by luminescence imaging weekly, and clinical activity was assessed daily.

After 5 weeks of therapy, animals were imaged by fluorescent luciferin imaging, and the fluorescence was quantified using Living Image® software. This allows for the quantification of mouse tumor burden. The results are shown in FIG. 3. Animals that received only imatinib (50 mg/kg daily) had reduced tumor burden compared to control animals that received neither imatinib nor anti-WT-1 antibody. Animals that received 100 μg of anti-WT-1 antibody twice a week for 5 weeks were much improved over control mice and imatinib-treated mice. The largest reduction of tumor cells was found in animals that received the combination of anti-WT-1 antibody and imatinib (Group D). These animals also showed reduced growth of tumor, evident from their previous day of imaging a week earlier. (FIG. 2)

### Example 5

### Therapeutic Effects of Dasatinib Plus Anti-WT-1/HLA Antibody (ESKM) in a Human Leukemia Xenograft NSG Model

Three million BV173 human leukemia cells were injected IV by tail vein into NSG mice. On day 6, tumor engraftment was confirmed by firefly luciferase imaging in all mice that were to be treated; mice were then randomly divided into five different treatment groups (A, B, C, D, and E). Immediately after imaging on day 6, therapy was initiated with anti-WT-1 antibody ESKM 100 μg administered by intraperitoneal (IP) injection twice weekly. Dasatinib was also administered by IP injection at 40 mg/kg daily. Since dasatinib is not soluble in aqueous solution, it was administered dissolved in 50 μL DMSO. Group A: No therapy; Group B: DMSO only (vehicle control); Group C: dasatinib treatment only; Group D: ESK treatment only; Group E: combination of both dasatinib daily and ESK twice weekly. After 7 days of therapy, it was noted that the mice treated with dasatinib looked ill with significant weight loss. The dose was decreased to 20 mg/kg. The mice continued to be in poor health, with 1 death, and on day 11 of therapy dasatinib was discontinued due to toxicity. ESK antibody continued to be administered for the full 4 week treatment cycle. Tumor growth continued to be assessed by luminescence imaging weekly.

After 4 weeks of therapy, animals were imaged by fluorescent luciferin imaging, and the fluorescence was quantified using Living Image® software, quantifying mouse tumor burden. The results are shown in FIG. 6. Animals that received only dasatinib initially had clearance of tumor, but relapsed by week 4 of therapy. Animals that received 100 μg of anti-WT-1 antibody twice a week for 4 weeks were much improved over control mice, though they had increased tumor burden compared to the dasatinib only mice. Of the mice that received combination of dasatinib and ESK, one mouse had intracranial tumor relapse, and three others remain tumor free. The fifth mouse died from dasatinib toxicity on day 8 of therapy.

### REFERENCES

1. Mundlos S, et al. Nuclear localization of the protein encoded by the Wilms' tumor gene WT-1 in embryonic and adult tissues. Development 1993; 119: 1329-41.
2. Keilholz U, et al. Wilms' tumor gene 1 (WT-1) in human neoplasia. Leukemia 2005; 19: 1318-1323.
3. Inoue K, et al. WT-1 as a new prognostic factor and a new marker for the detection of minimal residual disease in acute leukemia. Blood 1994; 84 (9): 3071-3079.
4. Ogawa H, et al. The usefulness of monitoring WT-1 gene transcripts for the prediction and management of relapse following allogeneic stem cell transplantation in acute type leukemia. Blood 2003; 101 (5): 1698-1704.
5. Yarnagarni T, et al. Growth Inhibition of Human Leukemic Cells by WT-1 (Wilms Tumor Gene) Antisense Oligodeoxynucleotides: Implications for the Involvement of WT-1 in Leukemogenesis. Blood 1996; 87: 2878-2884.
6. Bellantuono I, et al. Two distinct HLA-A0201-presented epitopes of the Wilms tumor antigen 1 can function as targets for leukemia-reactive CTL. Blood 2002; 100 (10): 3835-3837.
7. Gaiger A, et al. WT-1-specific serum antibodies in patients with leukemia. Clin. Cancer Res. 2001; 7 (suppl 3): 761-765.
8. Oka Y, et al. WT-1 peptide cancer vaccine for patients with hematopoietic malignancies and solid cancers. The Scientific World Journal 2007; 7: 649-665.
9. Kobayashi H, et al. Defining MHC class II T helper epitopes from WT-1 antigen. Cancer Immunol. Immunother. 2006; 55 (7): 850-860.
10. Pinilla-Ibarz J, et al. Improved human T-cell responses against synthetic HLA-A0201 analog peptides derived from the WT-1 oncoprotein. Leukemia 2006; 20 (11): 2025-2033.
11. May R J, et al. Peptide epitopes from the Wilms tumor 1 oncoprotein stimulate CD4+ and CD8+ T cells that recognize and kill human malignant mesothelioma tumor cells. Clin Cancer Res. 2007; 13:4547-4555.
12. Keiholz U, et al. A clinical and immunologic phase 2 trial of Wils tumor gene product (WT-1) peptide vaccination in patients with AML and MDS. Blood 2009; 113: 6541-6548.
13. Rezwani K, et al. Leukemia-associated antigen-specific T-cell responses following combined PR1 and WT-1 peptide vaccination in patients with myeloid malignancies. Blood 2008; 111 (1): 236-242.
14. Maslak P, et al. Vaccination with synthetic analog peptides derived from WT-1 oncoprotein induces T cell responses in patients with complete remission from acute myeloid leukemia. Blood 2010; Accpt Minor rev.
15. Krug L M, et al. WT-1 peptide vaccinations induce CD4 and CD8 T cell immune responses in patients with mesothelioma and non-small cell lung cancer. Cancer Immunol Immunother 2010; in revision.
16. Morris E, et al. Generation of tumor-specific T-cell therapies. Blood Reviews 2006; 20: 61-69.
17. Houghton A N et al. Monoclonal antibody therapies—a “constant” threat to cancer. Nat Med 2000; 6:373-374.
18. Miederer M, et al. Realizing the potential of the Actinium-225 radionuclide generator in targeted alpha particle therapy applications. Adv Drug Deliv Rev 2008; 60 (12): 1371-1382.
19. Noy R, T-cell-receptor-like antibodies: novel reagents for clinical cancer immunology and immunotherapy. Expert Rev Anticancer Ther 2005: 5 (3): 523-536.
20. Chames P, et al. Direct selection of a human antibody fragment directed against the tumor T-cell epitope HLA-A1-MAGE-A1 from a nonimmunized phage-Fab library. Proc Nalt Acad Sci USA 2000; 97: 7969-7974.
21. Held G, et al. Dissecting cytotoxic T cell responses towards the NY-ESO-1 protein by peptide/MHC-specific antibody fragments. Eur J Immunol. 2004: 34:2919-2929.
22. Lev A, et al. Isolation and characterization of human recombinant antibodies endowed with the antigen-specific, major histocompatibility complex-restricted specificity of T cells directed toward the widely expressed tumor T cell-epitopes of the telomerase catalytic subunit. Cancer Res 2002; 62: 3184-3194.
23. Klechevsky E, et al. Antitumor activity of immunotoxins with T-cell receptor-like specificity against human melanoma xenografts. Cancer Res 2008; 68 (15): 6360-6367.
24. Azinovic I, et al. Survival benefit associated with human anti-mouse antibody (HAMA) in patients with B-cell malignancies. Cancer Immunol Immunother 2006; 55(12):1451-8.
25. Tjandra J J, et al. Development of human anti-murine antibody (HAMA) response in patients. Immunol Cell Biol 1990; 68(6):367-76.
26. Riechmann L, et al. Reshaping human antibodies for therapy. Nature 1988; 332 (6162): 332:323.
27. Queen C, et al. A humanized antibody that binds to the interleukin 2 receptor. Proc Natl Acad Sci USA 1989; 86 (24): 10029-33.
28. Gerd R, et al. Serological Analysis of Human Anti-Human Antibody Responses in Colon Cancer Patients Treated with Repeated Doses of Humanized Monoclonal Antibody A33. Cancer Res 2001; 61, 6851-6859.
29. Cheever M A, et al. The prioritization of cancer antigens: A national Cancer Institute pilot project for the acceleration of translational research. Clin Cancer Res 2009; 15 (17): 5323-5337.
30. Drakos E, et al. Differentival expression of WT-1 gene product in non-Hodgkin lymphomas. Appl Immunohistochem Mol Morphol 2005; 13 (2): 132-137.
31. Asemissen A M, et al. Identification of a highly immunogenic HLA-A*01-binding T cell epitope of WT-1. Clin Cancer Res 2006; 12 (24):7476-7482.
32. Tomimatsu K, et al. Production of human monoclonal antibodies against FceRIa by a method combining in vitro immunization with phage display. Biosci Biotechnol Biochem 2009; 73 (7): 1465-1469.
33. Lidija P, et al. An integrated vector system for the eukaryotic expression of antibodies or their fragments after selection from phage display libraries. Gene 1997; 187(1): 9-18.
34. Lisa J H, et al. Crystallographic structure of an intact IgG1 monoclonal antibody. Journal of Molecular Biology 1998; 275 (5): 861-872.
35. Yasmina N A, et al. Probing the binding mechanism and affinity of tanezumab, a recombinant humanized anti-NGF monoclonal antibody, using a repertoire of biosensors. Protein Science 2008; 17(8): 1326-1335.
36. Roberts W K, et al. Vaccination with CD20 peptides induces a biologically active, specific immune response in mice. Blood 2002: 99 (10): 3748-3755.
37. Caron P C, Class K, Laird W, Co M S, Queen C, Scheinberg D A. Engineered humanized dimeric forms of IgG are more effective antibodies. J Exp Med 176:1191-1195. 1992.
38. McDevitt M, et al. Tumor targeting with antibody-functionalized, radiolabeled carbon nanotubes. J. Nuclear Med 2207; 48 (7))1180-1189.
39. Xue S A, et al. Development of a Wilms' tumor-specific T-cell receptor for clinical trials: engineered patient's T cells can eliminate autologous leukemia blasts in NOD/SCID mice. Haematologica 2010; 95 (1): 126-134.
40. McDevitt M R, et al. Tumor therapy with targeted atomic nanogenerators. Science 2001; 294 (5546): 1537-1540.
41. Borchardt P E, et al. Targeted Actinium-225 in vivo generators for therapy of ovarian cancer. Cancer Res 2003; 63: 5084-5090.
42. Singh Jaggi J, et al. Selective alpha-particle mediated depletion of tumor vasculature with vascular normalization. Plos One 2007; 2 (3): e267.
43. Yan W, et al. Enhancing antibody Fc heterodimer formation through electrostatic steering effects. J. Biol. Chem. 2010; 285: 19637-19646.
44. Rossi E A, et al. Stably tethered multi-functional structures of defined composition made by the dock and lock method for use in cancer targeting. Proc Natl Aca Sci USA 2006; 103:6841-6.
45. Ryutaro A, et al. Cytotoxic enhancement of a bispecific diabody by format conversion to tandem single-chain variable fragment (taFv). J Biol Chem 2011; 286: 1812-1818.
46. Anja L, et al. A recombinant bispecific single-chain antibody, CD19×CD3, induces rapid and high lymphoma-directed cytotoxicity by unstimulated T lymphocytes. Blood 2000; 95(6): 2098-2103.
47. Weiner G J, et al. The role of T cell activation in anti-CD3×antitumor bispecific antibody therapy. J. Immunology 1994; 152(5): 2385-2392.
48. Dafne M, et al. Improved pharmacokinetics of recombinant bispecific antibody molecules by fusion to human serum albumin. J Biol Chem 2007; 282: 12650-12660.
49. Liu C, et al. Modified host cells and uses thereof, PCT/US2010/0081195.
50. Francisco J, et al. Neutrophils Contribute to the Biological Antitumor Activity of Rituximab in a Non-Hodgkin's Lymphoma Severe Combined Immunodeficiency Mouse Model. Clin Cancer Res 2003; 9: 5866.
51. Kavita M, et al. Selective blockade of inhibitory Fc receptor enables human dendritic cell maturation with IL-12p70 production and immunity to antibody-coated tumor cells. Proc natl Aca Sci USA 2005; 102(8): 2910-2915.
52. Raphael A, et al. Inhibitory Fc receptors modulate in vivo cytoxicity against tumor targets. Nature Medicine 2000; 6:443-446.
53. Milenic E D. Monoclonal antibody-based therapy strategies: providing options for the cancer patient. Curr Pharm Des. 2002; 8: 1794-1764.
54. Grillo-Lopez A J. Anti-CD20 mAbs: modifying therapeutic strategies and outcomes in the treatment of lymphoma patients. Expert Rev Anticancer Ther. 2002: 2 (3): 323-329.
55. Jones K L & Buzdar A U. Evolving novel anti-Her2 strategies. Lancet Oncol. 2009: 10 (12): 1179-1187.
56. Reddy M M, Deshpande A & Sattler M. targeting JAK2 in the therapy of myeloproliferative neoplasms. Exper Opin Ther targets 2012:3:313-324.
57. Takeuchi K & Ito F. Receptor tyrosine kinases and targeted cancer therapeutics. Biol Pharm Bull. 2011; 34 (12) 1774-1780.
58. Roychowdhury S & Talpaz M. Managing resistance in chronic myeloid leukemia. Blood Rev. 2011; (6): 279-290.
59. Konnig R. Interactions between MHC molecules and co-receptors of the TCR. Curr Opin Immunol 2002: 14 (1) 75-83.
60. Sergeeva A, Alatrash G, He H, Ruisaard K, Lu S, Wygant J, McIntyre B W, Ma Q, Li D, St John L, Clise-Dwyer K & Molldrem J J. An anti-PR1/HLA-A2 T-cell receptor-like antibody mediated complement-dependent cytotoxicity against acute myeloid leukemia progenitor cells. Blood 2011; 117 (16): 4262-4272).
61. Takigawa N, Kiura K & Kishimoto T. Medical Treatment of Mesothelioma: Anything New? Curr Oncol Rep 2011; DOI 10.1007/s11912-011-0172-1.
62. Raja S, Murthy S C & Mason D P. Malignant Pleural Mesothelioma. Curr Oncol Rep 2011; DOI 10.1007/s11912-0177-9.
63. Gerber J M, Qin L, Kowalski J, Smith D, Griffin C A, Vala M S, Collector M I, Perkins B, Zahurak M, Matsui W, Gocke C D, Sharkis S, Levitsky H & Jones R J. Characterization of chronic myeloid leukemia stem cells. 2011; Am J Hematol. 86: 31-37.
64. Rezwani K, Yong A S, Savani B N, Mielke S, Keyvanfar K, Gostick E, Price D A, Douek D C & Barrett A J. Graft-versus-leukemia effects associated with detectable Wilms tumor-1 specific T lymphocytes after allogeneic stem-cell transplantation for acute lymphoblastic leukemia. Blood 2007: 110 (6): 1924-1932.
65. Persic L, Roberts A, Wilton J et al. An integrated vector system for the eukaryotic expression of antibodies or their fragments after selection from phage display libraries. Gene 1997; 187(1): 9-18.
66. Cheng L, Xiang J Y, Yan S et al. Modified host cells and uses thereof. PCT/US2010/0081195.
67. Lindmo T, Boven E, Cuttitta F, Fedorko J & Bunn P A Jr. Determination of the immunoreactive fraction of radiolabeled monoclonal antibodies by linear extrapolation to binding at infinite antigen excess. J Immunol Methods. 1984; 72 (1): 77-89.
68. Feng M, Zhang J L, Anver M, Hassan R & Ho M. In vivo imaging of human malignant mesothelioma growth orthotopically in the peritoneal cavity of nude mice. J Cancer 2011; 2: 123-131.
69. Demetri G D. Differential properties of current tyrosine kinase inhibitors in gastrointestinal stromal tumors. Semin Oncol 2011; 38 Suppl 1:S10-9.
70. Warnault P, Yasri A, Coisy-Quivy M, Chevé G, Boriés C, Fauvel B, Benhida R. Recent Advances in Drug Design of Epidermal Growth Factor Receptor Inhibitors. d Chem. 2013 Feb. 14 [Epub ahead of print].
71. Sivendran S, Liu Z, Portas L J Jr, Yu M, Hahn N, Sonpavde G, Oh W K, Galsky M D. Treatment-related mortality with vascular endothelial growth factor receptor tyrosine kinase inhibitor therapy in patients with advanced solid tumors: a meta-analysis. Cancer Treat Rev. 2012 November; 38(7):919-25.
72. Cabezón-Gutierrez L, Khosravi-Shahi P, Diaz Muñoz-de-la-Espada V M, Carrión-Galindo J R, Erañ a-Tomas I, Castro-Otero M. ALK-mutated non-small-cell lung cancer: a new strategy for cancer treatment. Lung. 2012 August; 190(4):381-8.
73. Barni S, Cabiddu M, Guarneri P, Lonati V, Petrelli F. The risk for anemia with targeted therapies for solid tumors. Oncologist. 2012; 17(5):715-24.
74. Dasanu C A, Padmanabhan P, Clark B A 3rd, Do C. Cardiovascular toxicity associated with small molecule tyrosine kinase inhibitors currently in clinical use. Expert Opin Drug Saf. 2012 May; 11(3):445-57.
75. Nakatsuka S, Oji Y, Horiuchi T, Kanda T, Kitagawa M, Takeuchi T, Kawano K, Kuwae Y, Yamauchi A, Okumura M, Kitamura Y, Oka Y, Kawase I, Sugiyama H, Aozasa K. Immunohistochemical detection of WT1 protein in a variety of cancer cells. Modern Pathology. 2006; 19:804-814.

What is claimed is:

1. A method for treating or inhibiting the proliferation of a WT-1 positive cancer, the method comprising administering to a subject in need thereof, a therapeutically effective amount of a tyrosine kinase inhibitor and a therapeutically effective amount of an anti-WT-1 antibody or antigen-binding fragment thereof.

2. The method of claim 1, wherein said WT-1 positive cancer is selected from the group consisting of chronic myelogenous leukemia (CML), acute myeloid leukemia (AML), acute lymphoblastic leukemia (ALL), and myelodysplastic syndrome (MDS), gastrointestinal stromal tumor, ovarian cancer, prostate cancer, soft tissue sarcoma, and malignant glioma.

3. The method of claim 1, wherein the tyrosine kinase inhibitor is selected from the group consisting of imatinib, dasatinib, nilotinib, bosutinib, ponatinib, bafetinib, erlotinib, gefitinib, lapatinib, sorafenib, and sunitinib.

4. The method of claim 1, wherein the tyrosine kinase inhibitor is imatinib or dasatinib or a pharmaceutically acceptable salt thereof.

5. The method of claim 5, wherein the pharmaceutically acceptable salt of imatinib is imatinib mesylate.

6. The method of claim 1, wherein said anti-WT-1 antibody is selected from the group consisting of:
(A) a antibody comprising a heavy chain (HC) variable region comprising HC-CDR1, HC-CDR2 and HC-CDR3; and a light chain (LC) variable region comprising LC-CDR1, LC-CDR2 and LC-CDR3, comprising amino acid sequences shown in Tables 1-14 and FIGS. 7-10; or
(B) an antibody comprising V_{H} and V_{L} comprising first and second amino acid sequences from Tables 1-12; or
(C) an antibody comprising an scFv comprising an amino acid sequence from Tables 1-12.

7. The method of claim 1, wherein the anti-WT-1 antibody comprises a human variable region framework region.

8. The method of claim 1, wherein the anti-WT-1 antibody is fully human.

9. The method of claim 1, wherein the anti-WT-1 antibody, or antigen-binding portion thereof, specifically binds a WT-1 peptide in an HLA restricted manner.

10. The method of claim 1, wherein the anti-WT-1 antibody, or an antigen-binding portion thereof, binds to WT-1/HLA with a K_{D} of 1×10⁻⁸ M or less.

11. The method of claim 1, wherein the anti-WT-1 antibody, or an antigen-binding portion thereof, binds to WT-1/HLA with a K_{D} of about 1×10⁻¹¹ M to about 1×10⁻⁸ M.

12. The method of claim 1, wherein the anti-WT-1 antibody, or an antigen-binding portion thereof, induces antibody dependent cellular cytotoxicity (ADCC) against WT-1-positive cells.

13. The method of claim 1, wherein the anti-WT-1 antibody, or an antigen-binding portion thereof inhibits growth of WT-1 positive cells in vivo.

14. The method of claim 1, wherein the antigen-binding fragment of said antibody is an Fab, Fab′, F(ab′)₂, Fv or single chain Fv (scFv).


SEQUENCE LISTING (456 sequences)

SEQ ID NO: 1 - protein, 9 aa, Homo sapiens
RMFPNAPYL

SEQ ID NO: 2 - protein, 10 aa, Homo sapiens
GGTFSSYAIS

SEQ ID NO: 3 - protein, 17 aa, Homo sapiens
GIIPIFGTANYAQKFQG

SEQ ID NO: 4 - protein, 10 aa, Homo sapiens
RIPPYYGMDV

SEQ ID NO: 5 - DNA, 30 bp, Homo sapiens
ggaggcaccttcagcagctatgctatcagc

SEQ ID NO: 6 - DNA, 51 bp, Homo sapiens
gggatcatccctatctttggtacagcaaactacgcacagaagttccagggc

SEQ ID NO: 7 - DNA, 30 bp, Homo sapiens
cggattcccccgtactacggtatggacgtc

SEQ ID NO: 8 - protein, 13 aa, Homo sapiens
SGSSSNIGSNYVY

SEQ ID NO: 9 - protein, 7 aa, Homo sapiens
RSNQRPS

SEQ ID NO: 10 - protein, 11 aa, Homo sapiens
AAWDDSLNGVV

SEQ ID NO: 11 - DNA, 39 bp, Homo sapiens
tctggaagcagctccaacatcggaagtaattatgtatac

SEQ ID NO: 12 - DNA, 21 bp, Homo sapiens
aggagtaatcagcggccctca

SEQ ID NO: 13 - DNA, 33 bp, Homo sapiens
gcagcatgggatgacagcctgaatggtgtggta

SEQ ID NO: 14 - protein, 119 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 15 - DNA, 357 bp, Homo sapiens
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagacggattcccccgtactacggtatggacgtctggggccaagggaccacggtcaccgtctcctca

SEQ ID NO: 16 - protein, 111 aa, Homo sapiens
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLG

SEQ ID NO: 17 - DNA, 333 bp, Homo sapiens
cagactgtggtgactcagccaccctcagcgtctgggacccccgggcagagggtcaccatctcttgttctggaagcagctccaacatcggaagtaattatgtatactggtaccaacagctcccaggaacggcccccaaactcctcatctataggagtaatcagcggccctcaggggtccctgaccgattctctggctccaagtctggcacctcagcctccctggccatcagtgggccccggtccgtggatgaggctgattattactgtgcagcatgggatgacagcctgaatggtgtggtattcggcggagggaccaagctgaccgtcctaggt

SEQ ID NO: 18 - protein, 250 aa, Homo sapiens
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLGSRGGGGSGGGGSGGGSLEMAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 19 - DNA, 750 bp, Homo sapiens
cagactgtggtgactcagccaccctcagcgtctgggacccccgggcagagggtcaccatctcttgttctggaagcagctccaacatcggaagtaattatgtatactggtaccaacagctcccaggaacggcccccaaactcctcatctataggagtaatcagcggccctcaggggtccctgaccgattctctggctccaagtctggcacctcagcctccctggccatcagtgggccccggtccgtggatgaggctgattattactgtgcagcatgggatgacagcctgaatggtgtggtattcggcggagggaccaagctgaccgtcctaggttctagaggtggtggtggtagcggcggcggcggctctggtggtggatccctcgagatggcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagacggattcccccgtactacggtatggacgtctggggccaagggaccacggtcaccgtctcctca

SEQ ID NO: 20 - protein, 12 aa, Homo sapiens
GDSVSSNSAAWN

SEQ ID NO: 21 - protein, 18 aa, Homo sapiens
RTYYGSKWYNDYAVSVKS

SEQ ID NO: 22 - protein, 9 aa, Homo sapiens
GRLGDAFDI

SEQ ID NO: 23 - DNA, 36 bp, Homo sapiens
ggggacagtgtctctagcaacagtgctgcttggaac

SEQ ID NO: 24 - DNA, 54 bp, Homo sapiens
aggacatactacgggtccaagtggtataatgattatgcagtatctgtgaaaagt

SEQ ID NO: 25 - DNA, 27 bp, Homo sapiens
ggtcgcttaggggatgcttttgatatc

SEQ ID NO: 26 - protein, 11 aa, Homo sapiens
RASQSISSYLN

SEQ ID NO: 27 - protein, 7 aa, Homo sapiens
AASSLQS

SEQ ID NO: 28 - protein, 9 aa, Homo sapiens
QQSYSTPLT

SEQ ID NO: 29 - DNA, 33 bp, Homo sapiens
cgggcaagtcagagcattagcagctatttaaat

SEQ ID NO: 30 - DNA, 21 bp, Homo sapiens
gctgcatccagtttgcaaagt

SEQ ID NO: 31 - DNA, 27 bp, Homo sapiens
caacagagttacagtacccctctcact

SEQ ID NO: 32 - protein, 121 aa, Homo sapiens
QVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 33 - DNA, 363 bp, Homo sapiens
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacgggtccaagtggtataatgattatgcagtatctgtgaaaagtcgaataaccatcaacccagacacatccaagaaccagttctccctgcagctgaactctgtgactcccgaggacacggctgtgtattactgtgcaagaggtcgcttaggggatgcttttgatatctggggccaagggacaatggtcaccgtctcttca

SEQ ID NO: 34 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKR

SEQ ID NO: 35 - DNA, 324 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggagggaccaaagtggatatcaaacgt

SEQ ID NO: 36 - protein, 250 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKRSRGGGGSGGGGSGGGGSLEMAQVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 37 - DNA, 750 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggagggaccaaagtggatatcaaacgttctagaggtggtggtggtagcggcggcggcggctctggtggtggtggatccctcgagatggcccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacgggtccaagtggtataatgattatgcagtatctgtgaaaagtcgaataaccatcaacccagacacatccaagaaccagttctccctgcagctgaactctgtgactcccgaggacacggctgtgtattactgtgcaagaggtcgcttaggggatgcttttgatatctggggccaagggacaatggtcaccgtctcttca

SEQ ID NO: 38 - protein, 10 aa, Homo sapiens
GYSFTNFWIS

SEQ ID NO: 39 - protein, 17 aa, Homo sapiens
RVDPGYSYSTYSPSFQG

SEQ ID NO: 40 - protein, 12 aa, Homo sapiens
VQYSGYYDWFDP

SEQ ID NO: 41 - DNA, 30 bp, Homo sapiens
ggatacagcttcaccaacttctggatcagc

SEQ ID NO: 42 - DNA, 51 bp, Homo sapiens
agggttgatcctggctactcttatagcacctacagcccgtccttccaaggc

SEQ ID NO: 43 - DNA, 36 bp, Homo sapiens
gtacaatatagtggctactatgactggttcgacccc

SEQ ID NO: 44 - protein, 13 aa, Homo sapiens
SGSSSNIGSNTVN

SEQ ID NO: 45 - protein, 7 aa, Homo sapiens
SNNQRPS

SEQ ID NO: 46 - protein, 11 aa, Homo sapiens
AAWDDSLNGWV

SEQ ID NO: 47 - DNA, 39 bp, Homo sapiens
tctggaagcagctccaacatcggaagtaatactgtaaac

SEQ ID NO: 48 - DNA, 21 bp, Homo sapiens
agtaataatcagcggccctca

SEQ ID NO: 49 - DNA, 33 bp, Homo sapiens
gcagcatgggatgacagcctgaatggttgggtg

SEQ ID NO: 50 - protein, 121 aa, Homo sapiens
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 51 - DNA, 363 bp, Homo sapiens
cagatgcagctggtgcagtccggagcagaggtgaaagagcccggggagtctctgaggatctcctgtaagggttctggatacagcttcaccaacttctggatcagctgggtgcgccagatgcccgggaaaggcctggagtggatggggagggttgatcctggctactcttatagcacctacagcccgtccttccaaggccacgtcaccatctcagctgacaagtctaccagcactgcctacctgcagtggaacagcctgaaggcctcggacaccgccatgtattactgtgcgagagtacaatatagtggctactatgactggttcgacccctggggccagggaaccctggtcaccgtctcctca

SEQ ID NO: 52 - protein, 111 aa, Homo sapiens
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLG

SEQ ID NO: 53 - DNA, 333 bp, Homo sapiens
caggctgtggtgactcagccaccctcagcgtctgggacccccgggcagagggtcaccatctcttgttctggaagcagctccaacatcggaagtaatactgtaaactggtaccagcaggtcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggt

SEQ ID NO: 54 - protein, 253 aa, Homo sapiens
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGSRGGGGSGGGGSGGGGSLEMAQMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 55 - DNA, 759 bp, Homo sapiens
caggctgtggtgactcagccaccctcagcgtctgggacccccgggcagagggtcaccatctcttgttctggaagcagctccaacatcggaagtaatactgtaaactggtaccagcaggtcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggcggagggaccaagctgaccgtcctaggttctagaggtggtggtggtagcggcggcggcggctctggtggtggtggatccctcgagatggcccagatgcagctggtgcagtccggagcagaggtgaaagagcccggggagtctctgaggatctcctgtaagggttctggatacagcttcaccaacttctggatcagctgggtgcgccagatgcccgggaaaggcctggagtggatggggagggttgatcctggctactcttatagcacctacagcccgtccttccaaggccacgtcaccatctcagctgacaagtctaccagcactgcctacctgcagtggaacagcctgaaggcctcggacaccgccatgtattactgtgcgagagtacaatatagtggctactatgactggttcgacccctggggccagggaaccctggtcaccgtctcctca

SEQ ID NO: 56 - protein, 10 aa, Homo sapiens
GYNFSNKWIG

SEQ ID NO: 57 - protein, 17 aa, Homo sapiens
IIYPGYSDITYSPSFQG

SEQ ID NO: 58 - protein, 9 aa, Homo sapiens
HTALAGFDY

SEQ ID NO: 59 - DNA, 30 bp, Homo sapiens
ggctacaactttagcaacaagtggatcggc

SEQ ID NO: 60 - DNA, 51 bp, Homo sapiens
atcatctatcccggttactcggacatcacctacagcccgtccttccaaggc

SEQ ID NO: 61 - DNA, 27 bp, Homo sapiens
cacacagctttggccggctttgactac

SEQ ID NO: 62 - protein, 11 aa, Homo sapiens
RASQNINKWLA

SEQ ID NO: 63 - protein, 7 aa, Homo sapiens
KASSLES

SEQ ID NO: 64 - protein, 8 aa, Homo sapiens
QQYNSYAT

SEQ ID NO: 65 - DNA, 33 bp, Homo sapiens
cgggccagtcagaatatcaataagtggctggcc

SEQ ID NO: 66 - DNA, 21 bp, Homo sapiens
aaggcgtctagtttagaaagt

SEQ ID NO: 67 - DNA, 24 bp, Homo sapiens
caacaatataatagttatgcgacg

SEQ ID NO: 68 - protein, 118 aa, Homo sapiens
QVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 69 - DNA, 354 bp, Homo sapiens
caggtgcagctggtgcagtctggagcagaggtgaaaaagcccggagagtctctgaagatctcctgtaagggttctggctacaactttagcaacaagtggatcggctgggtgcgccaattgcccgggagaggcctggagtggatagcaatcatctatcccggttactcggacatcacctacagcccgtccttccaaggccgcgtcaccatctccgccgacacgtccattaacaccgcctacctgcactggcacagcctgaaggcctcggacaccgccatgtattattgtgtgcgacacacagctttggccggctttgactactggggcctgggcaccctggtcaccgtctcctca

SEQ ID NO: 70 - protein, 107 aa, Homo sapiens
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKR

SEQ ID NO: 71 - DNA, 321 bp, Homo sapiens
gacatccagatgacccagtctccttccaccctgtctgcatctgtaggagacagagtcacaatcacttgccgggccagtcagaatatcaataagtggctggcctggtatcagcagagaccagggaaagcccctcagctcctgatctataaggcgtctagtttagaaagtggggtcccatctaggttcagcggcagtggatctgggacagaatacactctcaccatcagcagcctgcagcctgatgattttgcaacttattactgccaacaatataatagttatgcgacgttcggccaagggaccaaggtggaaatcaaacgt

SEQ ID NO: 72 - protein, 246 aa, Homo sapiens
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKRSRGGGGSGGGGSGGGGSLEMAQVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 73 - DNA, 738 bp, Homo sapiens
gacatccagatgacccagtctccttccaccctgtctgcatctgtaggagacagagtcacaatcacttgccgggccagtcagaatatcaataagtggctggcctggtatcagcagagaccagggaaagcccctcagctcctgatctataaggcgtctagtttagaaagtggggtcccatctaggttcagcggcagtggatctgggacagaatacactctcaccatcagcagcctgcagcctgatgattttgcaacttattactgccaacaatataatagttatgcgacgttcggccaagggaccaaggtggaaatcaaacgttctagaggtggtggtggtagcggcggcggcggctctggtggtggtggatccctcgagatggcccaggtgcagctggtgcagtctggagcagaggtgaaaaagcccggagagtctctgaagatctcctgtaagggttctggctacaactttagcaacaagtggatcggctgggtgcgccaattgcccgggagaggcctggagtggatagcaatcatctatcccggttactcggacatcacctacagcccgtccttccaaggccgcgtcaccatctccgccgacacgtccattaacaccgcctacctgcactggcacagcctgaaggcctcggacaccgccatgtattattgtgtgcgacacacagctttggccggctttgactactggggcctgggcaccctggtcaccgtctcctca

SEQ ID NO: 74 - protein, 10 aa, Homo sapiens
GFTFDDYGMS

SEQ ID NO: 75 - protein, 17 aa, Homo sapiens
GINWNGGSTGYADSVRG

SEQ ID NO: 76 - protein, 12 aa, Homo sapiens
ERGYGYHDPHDY

SEQ ID NO: 77 - DNA, 30 bp, Homo sapiens
gggttcacctttgatgattatggcatgagc

SEQ ID NO: 78 - DNA, 51 bp, Homo sapiens
ggtattaattggaatggtggtagcacaggttatgcagactctgtgaggggc

SEQ ID NO: 79 - DNA, 36 bp, Homo sapiens
gagcgtggctacgggtaccatgatccccatgactac

SEQ ID NO: 80 - protein, 11 aa, Homo sapiens
GRNNIGSKSVH

SEQ ID NO: 81 - protein, 7 aa, Homo sapiens
DDSDRPS

SEQ ID NO: 82 - protein, 11 aa, Homo sapiens
QVWDSSSDHVV

SEQ ID NO: 83 - DNA, 33 bp, Homo sapiens
gggagaaacaacattggaagtaaaagtgtgcac

SEQ ID NO: 84 - DNA, 21 bp, Homo sapiens
gatgatagcgaccggccctca

SEQ ID NO: 85 - DNA, 33 bp, Homo sapiens
caggtgtgggatagtagtagtgatcatgtggta

SEQ ID NO: 86 - protein, 121 aa, Homo sapiens
EVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 87 - DNA, 363 bp, Homo sapiens
gaagtgcagctggtgcagtctgggggaggtgtggtacggcctggggggtccctgagactctcctgtgcagcctctgggttcacctttgatgattatggcatgagctgggtccgccaagctccagggaaggggctggagtgggtctctggtattaattggaatggtggtagcacaggttatgcagactctgtgaggggccgattcaccatctccagagacaacgccaagaactccctgtatctgcaaatgaacagtctgagagccgaggacacggccttgtattactgtgcgagagagcgtggctacgggtaccatgatccccatgactactggggccaaggcaccctggtgaccgtctcctca

SEQ ID NO: 88 - protein, 109 aa, Homo sapiens
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLG

SEQ ID NO: 89 - DNA, 327 bp, Homo sapiens
cagtctgtcgtgacgcagccgccctcggtgtcagtggccccaggaaagacggccaggattacctgtgggagaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctaggt

SEQ ID NO: 90 - protein, 249 aa, Homo sapiens
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLGSRGGGGSGGGGSGGSLEMAEVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 91 - DNA, 747 bp, Homo sapiens
cagtctgtcgtgacgcagccgccctcggtgtcagtggccccaggaaagacggccaggattacctgtgggagaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctaggttctagaggtggtggtggtagcggcggcggcggctctggtggatccctcgagatggccgaagtgcagctggtgcagtctgggggaggtgtggtacggcctggggggtccctgagactctcctgtgcagcctctgggttcacctttgatgattatggcatgagctgggtccgccaagctccagggaaggggctggagtgggtctctggtattaattggaatggtggtagcacaggttatgcagactctgtgaggggccgattcaccatctccagagacaacgccaagaactccctgtatctgcaaatgaacagtctgagagccgaggacacggccttgtattactgtgcgagagagcgtggctacgggtaccatgatccccatgactactggggccaaggcaccctggtgaccgtctcctca

SEQ ID NO: 92 - protein, 10 aa, Homo sapiens
GFSVSGTYMG

SEQ ID NO: 93 - protein, 16 aa, Homo sapiens
LLYSGGGTYHPASLQG

SEQ ID NO: 94 - protein, 10 aa, Homo sapiens
GGAGGGHFDS

SEQ ID NO: 95 - DNA, 30 bp, Homo sapiens
gggttctccgtcagtggcacctacatgggc

SEQ ID NO: 96 - DNA, 48 bp, Homo sapiens
cttctttatagtggtggcggcacataccacccagcgtccctgcagggc

SEQ ID NO: 97 - DNA, 30 bp, Homo sapiens
ggaggggcaggaggtggccactttgactcc

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
AAWDDSLNGYV

SEQ ID NO: 101 - DNA, 42 bp, Homo sapiens
actgggagcagctccaacatcggggcaggttatgatgtacac

SEQ ID NO: 102 - DNA, 21 bp, Homo sapiens
ggtaacagcaatcggccctca

SEQ ID NO: 103 - DNA, 33 bp, Homo sapiens
gcagcatgggatgacagcctgaatggttatgtc

SEQ ID NO: 104 - protein, 118 aa, Homo sapiens
EVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 105 - DNA, 354 bp, Homo sapiens
gaggtgcagctggtggagaccggaggaggcttgctccagccgggggggtccctcagactctcctgtgcagcctctgggttctccgtcagtggcacctacatgggctgggtccgccaggctccagggaagggactggagtgggtcgcacttctttatagtggtggcggcacataccacccagcgtccctgcagggccgattcatcgtctccagagacagctccaagaatatggtctatcttcaaatgaatagcctgaaagccgaggacacggccgtctattactgtgcgaaaggaggggcaggaggtggccactttgactcctggggccaaggcaccctggtgaccgtctcctca

SEQ ID NO: 106 - protein, 112 aa, Homo sapiens
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLG

SEQ ID NO: 107 - DNA, 336 bp, Homo sapiens
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctgaccgtcctaggt

SEQ ID NO: 108 - protein, 251 aa, Homo sapiens
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLGSRGGGGSGGGGSGGGGSLEMAEVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 109 - DNA, 753 bp, Homo sapiens
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcagtgggctccagtctgaggatgaggctgattattactgtgcagcatgggatgacagcctgaatggttatgtcttcggaactgggaccaagctgaccgtcctaggttctagaggtggtggtggtagcggcggcggcggctctggtggtggtggatccctcgagatggccgaggtgcagctggtggagaccggaggaggcttgctccagccgggggggtccctcagactctcctgtgcagcctctgggttctccgtcagtggcacctacatgggctgggtccgccaggctccagggaagggactggagtgggtcgcacttctttatagtggtggcggcacataccacccagcgtccctgcagggccgattcatcgtctccagagacagctccaagaatatggtctatcttcaaatgaatagcctgaaagccgaggacacggccgtctattactgtgcgaaaggaggggcaggaggtggccactttgactcctggggccaaggcaccctggtgaccgtctcctca

SEQ ID NO: 110 - protein, 9 aa, Homo sapiens
AMFPNAPYL

SEQ ID NO: 111 - protein, 9 aa, Homo sapiens
RMAPNAPYL

SEQ ID NO: 112 - protein, 9 aa, Homo sapiens
RMFANAPYL

SEQ ID NO: 113 - protein, 9 aa, Homo sapiens
RMFPAAPYL

SEQ ID NO: 114 - protein, 9 aa, Homo sapiens
RMFPNAAYL

SEQ ID NO: 115 - protein, 9 aa, Homo sapiens
RMFPNAPAL

SEQ ID NO: 116 - protein, 9 aa, Homo sapiens
ILSLELMKL

SEQ ID NO: 117 - protein, 9 aa, Homo sapiens
QLQNPSYDK

SEQ ID NO: 118 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 119 - protein, 7 aa, Homo sapiens
SNAVAWN

SEQ ID NO: 120 - protein, 13 aa, Homo sapiens
RTYRGSTYYALSV

SEQ ID NO: 121 - protein, 8 aa, Homo sapiens
GSNSAFDF

SEQ ID NO: 122 - protein, 7 aa, Homo sapiens
SNSAAWN

SEQ ID NO: 123 - protein, 16 aa, Homo sapiens
RTYYGSKWYNDYAVSV

SEQ ID NO: 124 - protein, 9 aa, Homo sapiens
GRLGDAFDI

SEQ ID NO: 125 - protein, 7 aa, Homo sapiens
SDGAAWN

SEQ ID NO: 126 - protein, 16 aa, Homo sapiens
RTYYRSKWYNDYAVSV

SEQ ID NO: 127 - protein, 9 aa, Homo sapiens
GDYYYGMDV

SEQ ID NO: 128 - protein, 7 aa, Homo sapiens
SNAAAWN

SEQ ID NO: 129 - protein, 16 aa, Homo sapiens
RTYYGSKWYNDYAVSV

SEQ ID NO: 130 - protein, 5 aa, Homo sapiens
GAFDI

SEQ ID NO: 131 - protein, 5 aa, Homo sapiens
SYWIS

SEQ ID NO: 132 - protein, 17 aa, Homo sapiens
RIDPSDSYTNYSPSFQG

SEQ ID NO: 133 - protein, 9 aa, Homo sapiens
GDYDFYLDP

SEQ ID NO: 134 - protein, 5 aa, Homo sapiens
SYGIS

SEQ ID NO: 135 - protein, 17 aa, Homo sapiens
WISAYNGNTNYAQKLQG

SEQ ID NO: 136 - protein, 17 aa, Homo sapiens
DLYSSGWYESYYYGMDV

SEQ ID NO: 137 - protein, 5 aa, Homo sapiens
SYAIS

SEQ ID NO: 138 - protein, 17 aa, Homo sapiens
GIIPIFGTANYAQKFQG

SEQ ID NO: 139 - protein, 10 aa, Homo sapiens
RIPPYYGMDV

SEQ ID NO: 140 - protein, 17 aa, Homo sapiens
WISAHNGNTNYAQKLQG

SEQ ID NO: 141 - protein, 10 aa, Homo sapiens
DRVWFGDLSD

SEQ ID NO: 142 - protein, 17 aa, Homo sapiens
GIIPIFGTANYAQKEQG

SEQ ID NO: 143 - protein, 12 aa, Homo sapiens
NYDFWSGDAFDI

SEQ ID NO: 144 - protein, 12 aa, Homo sapiens
IPGTNYAQKFQG

SEQ ID NO: 145 - protein, 6 aa, Homo sapiens
FYGMDV

SEQ ID NO: 146 - protein, 5 aa, Homo sapiens
DYGMS

SEQ ID NO: 147 - protein, 15 aa, Homo sapiens
GINWNGGSTGYADSV

SEQ ID NO: 148 - protein, 12 aa, Homo sapiens
ERGYGYHDPHDY

SEQ ID NO: 149 - protein, 5 aa, Homo sapiens
NYTMN

SEQ ID NO: 150 - protein, 15 aa, Homo sapiens
SISLSGAYIYYADSL

SEQ ID NO: 151 - protein, 13 aa, Homo sapiens
EGYSSSVYDAFDL

SEQ ID NO: 152 - protein, 5 aa, Homo sapiens
SYGMH

SEQ ID NO: 153 - protein, 15 aa, Homo sapiens
GILSDGGKDYYVDSV

SEQ ID NO: 154 - protein, 12 aa, Homo sapiens
CSSNYGNDAFDI

SEQ ID NO: 155 - protein, 5 aa, Homo sapiens
TYSMN

SEQ ID NO: 156 - protein, 15 aa, Homo sapiens
SISSGAYSIFYADSV

SEQ ID NO: 157 - protein, 13 aa, Homo sapiens
DQYYGDKWDAFDI

SEQ ID NO: 158 - protein, 5 aa, Homo sapiens
SYGMN

SEQ ID NO: 159 - protein, 14 aa, Homo sapiens
SISSGGSIYYADSV

SEQ ID NO: 160 - protein, 9 aa, Homo sapiens
EYYWDAFDI

SEQ ID NO: 161 - protein, 14 aa, Homo sapiens
CSGSSSNIGSNTVN

SEQ ID NO: 162 - protein, 8 aa, Homo sapiens
SNNQRPSG

SEQ ID NO: 163 - protein, 13 aa, Homo sapiens
AAWDDSLNGWVFG

SEQ ID NO: 164 - protein, 13 aa, Homo sapiens
EAWDDSLKGPVFG

SEQ ID NO: 165 - protein, 15 aa, Homo sapiens
CTGSSSNIGAGYDVH

SEQ ID NO: 166 - protein, 8 aa, Homo sapiens
GNSNRPSG

SEQ ID NO: 167 - protein, 15 aa, Homo sapiens
QSYDSSLSADNYVFG

SEQ ID NO: 168 - protein, 14 aa, Homo sapiens
CSGSSSNIGRNIVN

SEQ ID NO: 169 - protein, 8 aa, Homo sapiens
SNIERPSG

SEQ ID NO: 170 - protein, 13 aa, Homo sapiens
ASWDDSLNGVLFG

SEQ ID NO: 171 - protein, 14 aa, Homo sapiens
CSGSRSNIASNGVG

SEQ ID NO: 172 - protein, 8 aa, Homo sapiens
KNDQRPSG

SEQ ID NO: 173 - protein, 14 aa, Homo sapiens
SAWDDSLDGHVVFG

SEQ ID NO: 174 - protein, 13 aa, Homo sapiens
AAWDDSLNGYVFG

SEQ ID NO: 175 - protein, 14 aa, Homo sapiens
CSGSSSNIGSSTVN

SEQ ID NO: 176 - protein, 8 aa, Homo sapiens
SNSQRPSG

SEQ ID NO: 177 - protein, 13 aa, Homo sapiens
AAWDDSLNGVVFG

SEQ ID NO: 178 - protein, 14 aa, Homo sapiens
CSGSSSNIGSNYVY

SEQ ID NO: 179 - protein, 8 aa, Homo sapiens
RSNQRPSG

SEQ ID NO: 180 - protein, 14 aa, Homo sapiens
CSGSSSNIGRNTVN

SEQ ID NO: 181 - protein, 14 aa, Homo sapiens
CSGSSSNIGNDYVS

SEQ ID NO: 182 - protein, 8 aa, Homo sapiens
DNNKRPSG

SEQ ID NO: 183 - protein, 13 aa, Homo sapiens
GTWDNSLSAWVFG

SEQ ID NO: 184 - protein, 14 aa, Homo sapiens
CSGSSSNIGSNSVY

SEQ ID NO: 185 - protein, 8 aa, Homo sapiens
NNNQRPSG

SEQ ID NO: 186 - protein, 13 aa, Homo sapiens
ATWDDSLSGWVFG

SEQ ID NO: 187 - protein, 8 aa, Homo sapiens
RNNQRPSG

SEQ ID NO: 188 - protein, 13 aa, Homo sapiens
AAWDDSLSAWVFG

SEQ ID NO: 189 - protein, 14 aa, Homo sapiens
CSGSTSNIGSYYVS

SEQ ID NO: 190 - protein, 8 aa, Homo sapiens
DNNNRPSG

SEQ ID NO: 191 - protein, 13 aa, Homo sapiens
GTWDSSLSAWVFG

SEQ ID NO: 192 - protein, 14 aa, Homo sapiens
CSGSSSNIGNNYVS

SEQ ID NO: 193 - protein, 14 aa, Homo sapiens
CSGSNSNIGTNTVT

SEQ ID NO: 194 - protein, 8 aa, Homo sapiens
SNFERPSG

SEQ ID NO: 195 - protein, 13 aa, Homo sapiens
SAWDDSFNGPVFG

SEQ ID NO: 196 - protein, 14 aa, Homo sapiens
CSGSSSNIGSNYVS

SEQ ID NO: 197 - protein, 13 aa, Homo sapiens
AAWDDGLRGYVFG

SEQ ID NO: 198 - protein, 11 aa, Homo sapiens
RASQSISSYLN

SEQ ID NO: 199 - protein, 7 aa, Homo sapiens
AASSLQS

SEQ ID NO: 200 - protein, 8 aa, Homo sapiens
QQSYSTPT

SEQ ID NO: 201 - protein, 11 aa, Homo sapiens
RASQGISNYLA

SEQ ID NO: 202 - protein, 7 aa, Homo sapiens
AASTLQS

SEQ ID NO: 203 - protein, 10 aa, Homo sapiens
QKYNSAPGVT

SEQ ID NO: 204 - protein, 11 aa, Homo sapiens
RASQSINGWLA

SEQ ID NO: 205 - protein, 7 aa, Homo sapiens
RASTLQS

SEQ ID NO: 206 - protein, 9 aa, Homo sapiens
QQSSSLPFT

SEQ ID NO: 207 - protein, 11 aa, Homo sapiens
RASQGISYYLA

SEQ ID NO: 208 - protein, 7 aa, Homo sapiens
AASTLKS

SEQ ID NO: 209 - protein, 9 aa, Homo sapiens
QQLNSYPLT

SEQ ID NO: 210 - protein, 11 aa, Homo sapiens
GGNNIGSKSVH

SEQ ID NO: 211 - protein, 7 aa, Homo sapiens
DDSDRPS

SEQ ID NO: 212 - protein, 11 aa, Homo sapiens
QVWDSSSDHPV

SEQ ID NO: 213 - protein, 11 aa, Homo sapiens
QVWDSSGDHPV

SEQ ID NO: 214 - protein, 7 aa, Homo sapiens
YDSDRPS

SEQ ID NO: 215 - protein, 11 aa, Homo sapiens
GGTNIGSRFVH

SEQ ID NO: 216 - protein, 11 aa, Homo sapiens
GGNNVESKSVH

SEQ ID NO: 217 - protein, 7 aa, Homo sapiens
YDRDRPS

SEQ ID NO: 218 - protein, 11 aa, Homo sapiens
EVWDSGSDHPV

SEQ ID NO: 219 - protein, 11 aa, Homo sapiens
GGKNIGSKSVH

SEQ ID NO: 220 - protein, 11 aa, Homo sapiens
QVWDSGSDHYV

SEQ ID NO: 221 - protein, 11 aa, Homo sapiens
QVWISSGDRVI

SEQ ID NO: 222 - protein, 11 aa, Homo sapiens
GGDNIGSQGVH

SEQ ID NO: 223 - protein, 7 aa, Homo sapiens
YDTDRPS

SEQ ID NO: 224 - protein, 11 aa, Homo sapiens
QVWGASSDHPV

SEQ ID NO: 225 - protein, 14 aa, Homo sapiens
TGTSSDVGGYNYVS

SEQ ID NO: 226 - protein, 7 aa, Homo sapiens
DVSKRPS

SEQ ID NO: 227 - protein, 10 aa, Homo sapiens
GIYTYSDSWV

SEQ ID NO: 228 - protein, 7 aa, Homo sapiens
DVGNRPS

SEQ ID NO: 229 - protein, 10 aa, Homo sapiens
SSYTSSSTRV

SEQ ID NO: 230 - protein, 14 aa, Homo sapiens
TGTRSDVGLYNYVA

SEQ ID NO: 231 - protein, 7 aa, Homo sapiens
DVIYRPG

SEQ ID NO: 232 - protein, 10 aa, Homo sapiens
SSYTNTGTVL

SEQ ID NO: 233 - protein, 14 aa, Homo sapiens
TGTSSDFGDYDYVS

SEQ ID NO: 234 - protein, 7 aa, Homo sapiens
DVSDRPS

SEQ ID NO: 235 - protein, 12 aa, Homo sapiens
QSYDSSLSGSGV

SEQ ID NO: 236 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 105 aa, Homo sapiens
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 239 - protein, 9 aa, Homo sapiens
CMTWNQMNL

SEQ ID NO: 240 - protein, 10 aa, Homo sapiens
GYTLTELSMH

SEQ ID NO: 241 - protein, 17 aa, Homo sapiens
GFDPEDGETIYAQKFQG

SEQ ID NO: 242 - protein, 10 aa, Homo sapiens
SFYSYYGIDT

SEQ ID NO: 243 - DNA, 30 bp, Homo sapiens
ggatacaccctcactgaattatccatgcac

SEQ ID NO: 244 - DNA, 51 bp, Homo sapiens
ggttttgatcctgaagatggtgaaacaatctacgcacagaagttccagggc

SEQ ID NO: 245 - DNA, 30 bp, Homo sapiens
tctttctactcttactacggtatcgatact

SEQ ID NO: 246 - protein, 11 aa, Homo sapiens
QGDSLRRYYAS

SEQ ID NO: 247 - protein, 7 aa, Homo sapiens
ANNNRPS

SEQ ID NO: 248 - protein, 11 aa, Homo sapiens
NSRDISVNGWM

SEQ ID NO: 249 - DNA, 33 bp, Homo sapiens
caaggagacagcctcagaaggtattatgcaagc

SEQ ID NO: 250 - DNA, 21 bp, Homo sapiens
gctaataacaatcggccctca

SEQ ID NO: 251 - DNA, 33 bp, Homo sapiens
aactcccgggacatcagtgttaacggttggatg

SEQ ID NO: 252 - protein, 119 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCARSFYSYYGIDTWGQGTLVTVSS

SEQ ID NO: 253 - DNA, 357 bp, Homo sapiens
caggtgcagctggtgcagtctggggctgaggtgaagaagcctggggcctcagtgaaggtctcctgcaaggtttccggatacaccctcactgaattatccatgcactgggtgcggcaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcgcgctctttctactcttactacggtatcgatacttggggtcaaggtactctggtgaccgtctcctca

SEQ ID NO: 254 - protein, 109 aa, Homo sapiens
SSELTQDPAVSVALGQTVRITCQGDSLRRYYASWYQQKPGQAPVLVIYANNNRPSGIPDRFSGSSSGNTASLTITGAQAEDEADYYCNSRDISVNGWMFGGGTKLTVLG

SEQ ID NO: 255 - DNA, 327 bp, Homo sapiens
tcttctgagctgactcaggaccctgctgtgtctgtggccttgggacagacagtcaggatcacatgccaaggagacagcctcagaaggtattatgcaagctggtaccagcagaagccaggacaggcccctgtacttgtcatctatgctaataacaatcggccctcagggatcccagaccgattctctggctccagctcaggaaacacagcttccttgaccatcactggggctcaggcggaggatgaggctgactattattgtaactcccgggacatcagtgttaacggttggatgttcggcggagggaccaagctgaccgtcctaggt

SEQ ID NO: 256 - protein, 249 aa, Homo sapiens
SSELTQDPAVSVALGQTVRITCQGDSLRRYYASWYQQKPGQAPVLVIYANNNRPSGIPDRFSGSSSGNTASLTITGAQAEDEADYYCNSRDISVNGWMFGGGTKLTVLGSRGGGGSGGGGSGGGGSLEMAQVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCARSFYSYYGIDTWGQGTLVTVSS

SEQ ID NO: 257 - DNA, 747 bp, Homo sapiens
tcttctgagctgactcaggaccctgctgtgtctgtggccttgggacagacagtcaggatcacatgccaaggagacagcctcagaaggtattatgcaagctggtaccagcagaagccaggacaggcccctgtacttgtcatctatgctaataacaatcggccctcagggatcccagaccgattctctggctccagctcaggaaacacagcttccttgaccatcactggggctcaggcggaggatgaggctgactattattgtaactcccgggacatcagtgttaacggttggatgttcggcggagggaccaagctgaccgtcctaggttctagaggtggtggtggtagcggcggcggcggctctggtggtggtggatccctcgagatggcccaggtgcagctggtgcagtctggggctgaggtgaagaagcctggggcctcagtgaaggtctcctgcaaggtttccggatacaccctcactgaattatccatgcactgggtgcggcaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcgcgctctttctactcttactacggtatcgatacttggggtcaaggtactctggtgaccgtctcctca

SEQ ID NO: 258 - protein, 10 aa, Homo sapiens
GYTFTTYGMN

SEQ ID NO: 259 - protein, 17 aa, Homo sapiens
WINTNTGKPTYAQGFTG

SEQ ID NO: 260 - protein, 10 aa, Homo sapiens
GYYGWDYHDY

SEQ ID NO: 261 - DNA, 30 bp, Homo sapiens
ggatacaccttcactacctatggtatgaat

SEQ ID NO: 262 - DNA, 51 bp, Homo sapiens
tggatcaacaccaacactgggaagccaacgtatgcccagggcttcacagga

SEQ ID NO: 263 - DNA, 30 bp, Homo sapiens
ggttactacggttgggactaccatgattac

SEQ ID NO: 264 - protein, 11 aa, Homo sapiens
GGNNIGSKSVH

SEQ ID NO: 265 - protein, 7 aa, Homo sapiens
YDSDRPS

SEQ ID NO: 266 - protein, 13 aa, Homo sapiens
QVWDSSSDHSPYV

SEQ ID NO: 267 - DNA, 33 bp, Homo sapiens
gggggaaacaacattggaagtaaaagtgtgcac

SEQ ID NO: 268 - DNA, 21 bp, Homo sapiens
tatgatagcgaccggccctca

SEQ ID NO: 269 - DNA, 39 bp, Homo sapiens
caggtgtgggatagtagtagtgatcattccccttatgtc

SEQ ID NO: 270 - protein, 119 aa, Homo sapiens
QVQLVQSGSELKKPGASVKVSCKASGYTFTTYGMNWVRQAPGQGLEWMGWINTNTGKPTYAQGFTGRFVFSLDASVSTAYLQISGLKADDTAVYYCARGYYGWDYHDYWGQGTLVTVSS

SEQ ID NO: 271 - DNA, 357 bp, Homo sapiens
caggtgcagctggtgcagtctgggtctgagttgaagaagcctggggcctcagtgaaggtttcctgcaaggcttctggatacaccttcactacctatggtatgaattgggtgcgacaggcccctggacaagggcttgagtggatgggatggatcaacaccaacactgggaagccaacgtatgcccagggcttcacaggacggtttgtcttctccttggacgcctctgtcagcacggcatatctgcagatcagcggcctaaaggctgacgacactgccgtgtattactgtgcgcgcggttactacggttgggactaccatgattactggggtcaaggtactctggtgaccgtctcctca

SEQ ID NO: 272 - protein, 111 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHSPYVFGTGTKLTVLG

SEQ ID NO: 273 - DNA, 333 bp, Homo sapiens
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcattccccttatgtcttcggaactgggaccaagctgaccgtcctaggt

SEQ ID NO: 274 - protein, 251 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHSPYVFGTGTKLTVLGSRGGGGSGGGGSGGGGSLEMAQVQLVQSGSELKKPGASVKVSCKASGYTFTTYGMNWVRQAPGQGLEWMGWINTNTGKPTYAQGFTGRFVFSLDASVSTAYLQISGLKADDTAVYYCARGYYGWDYHDYWGQGTLVTVSS

SEQ ID NO: 275 - DNA, 753 bp, Homo sapiens
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcattccccttatgtcttcggaactgggaccaagctgaccgtcctaggttctagaggtggtggtggtagcggcggcggcggctctggtggtggtggatccctcgagatggcccaggtgcagctggtgcagtctgggtctgagttgaagaagcctggggcctcagtgaaggtttcctgcaaggcttctggatacaccttcactacctatggtatgaattgggtgcgacaggcccctggacaagggcttgagtggatgggatggatcaacaccaacactgggaagccaacgtatgcccagggcttcacaggacggtttgtcttctccttggacgcctctgtcagcacggcatatctgcagatcagcggcctaaaggctgacgacactgccgtgtattactgtgcgcgcggttactacggttgggactaccatgattactggggtcaaggtactctggtgaccgtctcctca

SEQ ID NO: 276 - protein, 10 aa, Homo sapiens
GGTFSSYAIS

SEQ ID NO: 277 - protein, 17 aa, Homo sapiens
GIIPIFGTANYAQKFQG

SEQ ID NO: 278 - protein, 9 aa, Homo sapiens
WFYMQAGDH

SEQ ID NO: 279 - DNA, 30 bp, Homo sapiens; identical to SEQ ID NO: 5

SEQ ID NO: 280 - DNA, 51 bp, Homo sapiens; identical to SEQ ID NO: 6

SEQ ID NO: 281 - DNA, 27 bp, Homo sapiens
tggttctacatgcaggctggtgatcat

SEQ ID NO: 282 - protein, 14 aa, Homo sapiens
TGSSSDVGTYNYDS

SEQ ID NO: 283 - protein, 7 aa, Homo sapiens
DVSERPS

SEQ ID NO: 284 - protein, 10 aa, Homo sapiens
SSFAASSPWL

SEQ ID NO: 285 - DNA, 42 bp, Homo sapiens
actggaagcagcagtgatgttggtacttataactatgactct

SEQ ID NO: 286 - DNA, 21 bp, Homo sapiens
gatgtcagtgagcggccctca

SEQ ID NO: 287 - DNA, 30 bp, Homo sapiens
agctcatttgcagccagcagtccctggctg

SEQ ID NO: 288 - protein, 118 aa, Homo sapiens
QVQLVESGAEVKKPGSSVKVSCKASGGTFSSYAISWVRQAPGQGLEWMGGIIPIFGTANYAQKFQGRVTITADESTSTAYMELSSLRSEDTAVYYCARWFYMQAGDHWGQGTLVTVSS

SEQ ID NO: 289 - DNA, 354 bp, Homo sapiens
caggtgcagctggtggagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgcgctggttctacatgcaggctggtgatcattggggtcaaggtactctggtgaccgtctcctca

SEQ ID NO: 290 - protein, 111 aa, Homo sapiens
QAVLTQPASVSGSPGQSITISCTGSSSDVGTYNYDSWYQQHPGKAPKLMIYDVSERPSGVSNRFSGSKSGNTAFLTISGLQAEDEADYYCSSFAASSPWLFGGGTKVTVLG

SEQ ID NO: 291 - DNA, 333 bp, Homo sapiens
caggctgtgctgactcagcctgcctccgtgtctgggtctcctggacagtcgatcaccatctcctgcactggaagcagcagtgatgttggtacttataactatgactcttggtaccaacagcacccaggcaaagcccccaaactcatgatttatgatgtcagtgagcggccctcaggggtttctaatcgcttctccggctccaagtctggcaacacggccttcctgaccatctctgggctccaggctgaggacgaggctgattattactgcagctcatttgcagccagcagtccctggctgttcggcggagggaccaaggtcaccgtcctaggt

SEQ ID NO: 292 - protein, 250 aa, Homo sapiens
QAVLTQPASVSGSPGQSITISCTGSSSDVGTYNYDSWYQQHPGKAPKLMIYDVSERPSGVSNRFSGSKSGNTAFLTISGLQAEDEADYYCSSFAASSPWLFGGGTKVTVLGSRGGGGSGGGGSGGGGSLEMAQVQLVESGAEVKKPGSSVKVSCKASGGTFSSYAISWVRQAPGQGLEWMGGIIPIFGTANYAQKFQGRVTITADESTSTAYMELSSLRSEDTAVYYCARWFYMQAGDHWGQGTLVTVSS

SEQ ID NO: 293 - DNA, 750 bp, Homo sapiens
caggctgtgctgactcagcctgcctccgtgtctgggtctcctggacagtcgatcaccatctcctgcactggaagcagcagtgatgttggtacttataactatgactcttggtaccaacagcacccaggcaaagcccccaaactcatgatttatgatgtcagtgagcggccctcaggggtttctaatcgcttctccggctccaagtctggcaacacggccttcctgaccatctctgggctccaggctgaggacgaggctgattattactgcagctcatttgcagccagcagtccctggctgttcggcggagggaccaaggtcaccgtcctaggttctagaggtggtggtggtagcggcggcggcggctctggtggtggtggatccctcgagatggcccaggtgcagctggtggagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgcgctggttctacatgcaggctggtgatcattggggtcaaggtactctggtgaccgtctcctca

SEQ ID NO: 294 - protein, 10 aa, Homo sapiens
GFTFSSYGMN

SEQ ID NO: 295 - protein, 17 aa, Homo sapiens
SISSSSSYIYYADSVKG

SEQ ID NO: 296 - protein, 14 aa, Homo sapiens
IQDATGEEMILYDY

SEQ ID NO: 297 - DNA, 30 bp, Homo sapiens
ggattcaccttcagtagctatggcatgaac

SEQ ID NO: 298 - DNA, 51 bp, Homo sapiens
tccattagtagtagtagtagttacatatactacgcagactcagtgaagggc

SEQ ID NO: 299 - DNA, 42 bp, Homo sapiens
atccaggacgctactggtgaagaaatgatcctgtacgattac

SEQ ID NO: 300 - protein, 16 aa, Homo sapiens
RSSQSLVYSDGNTYLN

SEQ ID NO: 301 - protein, 7 aa, Homo sapiens
QVSKRDS

SEQ ID NO: 302 - protein, 8 aa, Homo sapiens
MQGSHLRT

SEQ ID NO: 303 - DNA, 48 bp, Homo sapiens
aggtctagtcaaagcctcgtatacagtgatggaaacacctatttgaat

SEQ ID NO: 304 - DNA, 21 bp, Homo sapiens
caggtttctaagcgggactct

SEQ ID NO: 305 - DNA, 24 bp, Homo sapiens
atgcaaggttcacacttgcggacg

SEQ ID NO: 306 - protein, 123 aa, Homo sapiens
QVQLVESGGGLVKPGGSLRLSCAASGFTFSSYGMNWVRQAPGKGLEWVSSISSSSSYIYYADSVKGRFTISRDNAKNSLYLQMNSLRAEDTAVYYCARIQDATGEEMILYDYWGQGTLVTVSS

SEQ ID NO: 307 - DNA, 369 bp, Homo sapiens
caggtgcagctggtggagtctgggggaggcctggtcaagcctggggggtccctgaggctctcctgtgcagcctctggattcaccttcagtagctatggcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgcgcatccaggacgctactggtgaagaaatgatcctgtacgattactggggtcaaggtactctggtgaccgtctcctca

SEQ ID NO: 308 - protein, 112 aa, Homo sapiens
EIVLTQSPLSLPVTLGQPASISCRSSQSLVYSDGNTYLNWFQQRPGQSPRRLIYQVSKRDSGVPDRFSGSGSGTDFTLKISRVEAEDVGMYYCMQGSHLRTFGQGTKVEIKR

SEQ ID NO: 309 - DNA, 333 bp, Homo sapiens
attgtgctgactcagtctccactctccctgcccgtcacccttggacagccggcctccatctcctgcaggtctagtcaaagcctcgtatacagtgatggaaacacctatttgaattggtttcagcagaggccaggccaatctccaaggcgcctaatttatcaggtttctaagcgggactctggggtcccagacagattcagcggcagtgggtcaggcactgatttcacactgaaaatcagcagggtggaggctgaggatgttgggatgtattactgcatgcaaggttcacacttgcggacgttcggccaagggaccaaggtggaaatcaaacgt

SEQ ID NO: 310 - protein, 256 aa, Homo sapiens
EIVLTQSPLSLPVTLGQPASISCRSSQSLVYSDGNTYLNWFQQRPGQSPRRLIYQVSKRDSGVPDRFSGSGSGTDFTLKISRVEAEDVGMYYCMQGSHLRTFGQGTKVEIKRSRGGGGSGGGGSGGGGSLEMAQVQLVESGGGLVKPGGSLRLSCAASGFTFSSYGMNWVRQAPGKGLEWVSSISSSSSYIYYADSVKGRFTISRDNAKNSLYLQMNSLRAEDTAVYYCARIQDATGEEMILYDYWGQGTLVTVSS

SEQ ID NO: 311 - DNA, 768 bp, Homo sapiens
gaaattgtgctgactcagtctccactctccctgcccgtcacccttggacagccggcctccatctcctgcaggtctagtcaaagcctcgtatacagtgatggaaacacctatttgaattggtttcagcagaggccaggccaatctccaaggcgcctaatttatcaggtttctaagcgggactctggggtcccagacagattcagcggcagtgggtcaggcactgatttcacactgaaaatcagcagggtggaggctgaggatgttgggatgtattactgcatgcaaggttcacacttgcggacgttcggccaagggaccaaggtggaaatcaaacgttctagaggtggtggtggtagcggcggcggcggctctggtggtggtggatccctcgagatggcccaggtgcagctggtggagtctgggggaggcctggtcaagcctggggggtccctgaggctctcctgtgcagcctctggattcaccttcagtagctatggcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgcgcatccaggacgctactggtgaagaaatgatcctgtacgattactggggtcaaggtactctggtgaccgtctcctca

SEQ ID NO: 312 - protein, 10 aa, Homo sapiens
GYTFTDYYIH

SEQ ID NO: 313 - protein, 17 aa, Homo sapiens
WMNPNSGNSVSAQKFQG

SEQ ID NO: 314 - protein, 14 aa, Homo sapiens
YQGSTWKYDSYGDL

SEQ ID NO: 315 - DNA, 30 bp, Homo sapiens
ggatacaccttcaccgactactatatacac

SEQ ID NO: 316 - DNA, 51 bp, Homo sapiens
tggatgaaccctaacagtgggaactcagtctctgcacagaagttccagggc

SEQ ID NO: 317 - DNA, 42 bp, Homo sapiens
taccagggttctacttggaaatacgactcttacggtgatctg

SEQ ID NO: 318 - protein, 11 aa, Homo sapiens
GGNEIGFNGVH

SEQ ID NO: 319 - protein, 7 aa, Homo sapiens
NNRVRPS

SEQ ID NO: 320 - protein, 11 aa, Homo sapiens
QVWVNPDNEYV

SEQ ID NO: 321 - DNA, 33 bp, Homo sapiens
gggggaaacgagattggatttaatggtgttcat

SEQ ID NO: 322 - DNA, 21 bp, Homo sapiens
aacaatagggtccggccctca

SEQ ID NO: 323 - DNA, 33 bp, Homo sapiens
caggtgtgggttaatcctgataatgaatatgtc

SEQ ID NO: 324 - protein, 123 aa, Homo sapiens
QVQLVQSGAEVKKPGASVKVSCKASGYTFTDYYIHWVRQAPGQGLEWMGWMNPNSGNSVSAQKFQGRVTMTRDTSINTAYMELSSLTSDDTAVYYCARYQGSTWKYDSYGDLWGQGTLVTVTS

SEQ ID NO: 325 - DNA, 369 bp, Homo sapiens
caggtccagctggtgcagtctggggctgaggtgaagaagcctggggcctcagtgaaggtctcctgcaaggcttctggatacaccttcaccgactactatatacactgggtgcggcaggcccctggacaagggctggagtggatgggatggatgaaccctaacagtgggaactcagtctctgcacagaagttccagggcagagtcaccatgaccagggatacctccataaacacagcctacatggagctgagcagcctgacatctgacgacacggccgtatattactgtgcgcgctaccagggttctacttggaaatacgactcttacggtgatctgtggggtcaaggtactctggtgaccgtcacctca

SEQ ID NO: 326 - protein, 109 aa, Homo sapiens
QAVLTQPPSVSVAPGETATVTCGGNEIGFNGVHWYKQKAGQAPLLVIYNNRVRPSGISERLSGSNSGNTATLTISRVEAGDEADYYCQVWVNPDNEYVFGSGTKVTVLG

SEQ ID NO: 327 - DNA, 327 bp, Homo sapiens
caggctgtgctgactcagccaccctcggtgtcagtggccccaggagagacggccactgttacctgtgggggaaacgagattggatttaatggtgttcattggtataagcagaaggcaggccaggcccctctgttggtcatctataacaatagggtccggccctcagggatctctgagcgactctctggctccaactctggtaacacggccaccctgaccatcagcagggtcgaagccggggatgaggccgactattactgtcaggtgtgggttaatcctgataatgaatatgtcttcggatcggggaccaaggtcaccgtcctaggt

SEQ ID NO: 328 - protein, 253 aa, Homo sapiens
QAVLTQPPSVSVAPGETATVTCGGNEIGFNGVHWYKQKAGQAPLLVIYNNRVRPSGISERLSGSNSGNTATLTISRVEAGDEADYYCQVWVNPDNEYVFGSGTKVTVLGSRGGGGSGGGGSGGGGSLEMAQVQLVQSGAEVKKPGASVKVSCKASGYTFTDYYIHWVRQAPGQGLEWMGWMNPNSGNSVSAQKFQGRVTMTRDTSINTAYMELSSLTSDDTAVYYCARYQGSTWKYDSYGDLWGQGTLVTVTS

SEQ ID NO: 329 - DNA, 759 bp, Homo sapiens
caggctgtgctgactcagccaccctcggtgtcagtggccccaggagagacggccactgttacctgtgggggaaacgagattggatttaatggtgttcattggtataagcagaaggcaggccaggcccctctgttggtcatctataacaatagggtccggccctcagggatctctgagcgactctctggctccaactctggtaacacggccaccctgaccatcagcagggtcgaagccggggatgaggccgactattactgtcaggtgtgggttaatcctgataatgaatatgtcttcggatcggggaccaaggtcaccgtcctaggttctagaggtggtggtggtagcggcggcggcggctctggtggtggtggatccctcgagatggcccaggtccagctggtgcagtctggggctgaggtgaagaagcctggggcctcagtgaaggtctcctgcaaggcttctggatacaccttcaccgactactatatacactgggtgcggcaggcccctggacaagggctggagtggatgggatggatgaaccctaacagtgggaactcagtctctgcacagaagttccagggcagagtcaccatgaccagggatacctccataaacacagcctacatggagctgagcagcctgacatctgacgacacggccgtatattactgtgcgcgctaccagggttctacttggaaatacgactcttacggtgatctgtggggtcaaggtactctggtgaccgtcacctca

SEQ ID NO: 330 - protein, 10 aa, Homo sapiens
GFTFSSYEMN

SEQ ID NO: 331 - protein, 17 aa, Homo sapiens
YISSSGSTIYYADSVKG

SEQ ID NO: 332 - protein, 13 aa, Homo sapiens
DWRSSYYYSQYDK

SEQ ID NO: 333 - DNA, 30 bp, Homo sapiens
ggattcaccttcagtagttatgaaatgaac

SEQ ID NO: 334 - DNA, 51 bp, Homo sapiens
tacattagtagtagtggtagtaccatatactacgcagactctgtgaagggc

SEQ ID NO: 335 - DNA, 39 bp, Homo sapiens
gactggcgttcttcttactactactctcagtacgataaa

SEQ ID NO: 336 - protein, 13 aa, Homo sapiens
TRSSGNIASNYVQ

SEQ ID NO: 337 - protein, 7 aa, Homo sapiens
ADNQRPS

SEQ ID NO: 338 - protein, 9 aa, Homo sapiens
QSYENNIHV

SEQ ID NO: 339 - DNA, 39 bp, Homo sapiens
acccgcagcagtggcaacattgccagcaactatgtgcag

SEQ ID NO: 340 - DNA, 21 bp, Homo sapiens
gcggacaaccaaagaccctct

SEQ ID NO: 341 - DNA, 27 bp, Homo sapiens
cagtcttatgaaaacaacattcacgtg

SEQ ID NO: 342 - protein, 122 aa, Homo sapiens
EVQLVESGGGLVQPGESLRLSCAASGFTFSSYEMNWVRQAPGKGLEWVSYISSSGSTIYYADSVKGRFTISRDNAKNSLYLQMNSLRAEDTAVYYCARDWRSSYYYSQYDKWGQGTLVTVSS

SEQ ID NO: 343 - DNA, 366 bp, Homo sapiens
gaggtgcagctggtggagtctgggggaggcttggtacagcctggagagtccctgagactctcctgtgcagcctctggattcaccttcagtagttatgaaatgaactgggttcgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtgtattactgtgcgcgcgactggcgttcttcttactactactctcagtacgataaatggggtcaaggtactctggtgaccgtctcctca

SEQ ID NO: 344 - protein, 111 aa, Homo sapiens
NFMLTQPHSVSESPGKTVSISCTRSSGNIASNYVQWYQHRPGRSPTTVIYADNQRPSGVPDRFSGSIDTSSNSASLTISGLRTEDEADYYCQSYENNIHVFGGGTKLTVLG

SEQ ID NO: 345 - DNA, 333 bp, Homo sapiens
aattttatgctgactcagccccactctgtgtcggagtctccggggaagacggtaagcatctcctgcacccgcagcagtggcaacattgccagcaactatgtgcagtggtaccaacaccgcccgggccgttcccccaccactgtgatctatgcggacaaccaaagaccctctggggtccctgatcgcttctctggctccatcgacacctcctccaactctgcctccctcaccatctctggactgaggactgaggacgaggctgactactactgtcagtcttatgaaaacaacattcacgtgttcggcggggggaccaagctgaccgtcctaggt

SEQ ID NO: 346 - protein, 254 aa, Homo sapiens
NFMLTQPHSVSESPGKTVSISCTRSSGNIASNYVQWYQHRPGRSPTTVIYADNQRPSGVPDRFSGSIDTSSNSASLTISGLRTEDEADYYCQSYENNIHVFGGGTKLTVLGSRGGGGSGGGGSGGGGSLEMAEVQLVESGGGLVQPGESLRLSCAASGFTFSSYEMNWVRQAPGKGLEWVSYISSSGSTIYYADSVKGRFTISRDNAKNSLYLQMNSLRAEDTAVYYCARDWRSSYYYSQYDKWGQGTLVTVSS

SEQ ID NO: 347 - DNA, 762 bp, Homo sapiens
aattttatgctgactcagccccactctgtgtcggagtctccggggaagacggtaagcatctcctgcacccgcagcagtggcaacattgccagcaactatgtgcagtggtaccaacaccgcccgggccgttcccccaccactgtgatctatgcggacaaccaaagaccctctggggtccctgatcgcttctctggctccatcgacacctcctccaactctgcctccctcaccatctctggactgaggactgaggacgaggctgactactactgtcagtcttatgaaaacaacattcacgtgttcggcggggggaccaagctgaccgtcctaggttctagaggtggtggtggtagcggcggcggcggctctggtggtggtggatccctcgagatggccgaggtgcagctggtggagtctgggggaggcttggtacagcctggagagtccctgagactctcctgtgcagcctctggattcaccttcagtagttatgaaatgaactgggttcgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtgtattactgtgcgcgcgactggcgttcttcttactactactctcagtacgataaatggggtcaaggtactctggtgaccgtctcctca

SEQ ID NO: 348 - protein, 9 aa, Homo sapiens
CMTWNQMNL

SEQ ID NO: 349 - protein, 10 aa, Homo sapiens
GYTLTELSMH

SEQ ID NO: 350 - protein, 17 aa, Homo sapiens
GFDPEDGETIYAQKFQG

SEQ ID NO: 351 - protein, 10 aa, Homo sapiens
SFYSYYGIDT

SEQ ID NO: 352 - DNA, 30 bp, Homo sapiens; identical to SEQ ID NO: 243

SEQ ID NO: 353 - DNA, 51 bp, Homo sapiens; identical to SEQ ID NO: 244

SEQ ID NO: 354 - DNA, 30 bp, Homo sapiens; identical to SEQ ID NO: 245

SEQ ID NO: 355 - protein, 11 aa, Homo sapiens
QGDSLRRYYAS

SEQ ID NO: 356 - protein, 7 aa, Homo sapiens
ANNNRPS

SEQ ID NO: 357 - protein, 11 aa, Homo sapiens
NSRDISVNGWM

SEQ ID NO: 358 - DNA, 33 bp, Homo sapiens; identical to SEQ ID NO: 249

SEQ ID NO: 359 - DNA, 21 bp, Homo sapiens
gctaataacaatcggccctca

SEQ ID NO: 360 - DNA, 33 bp, Homo sapiens; identical to SEQ ID NO: 251

SEQ ID NO: 361 - protein, 119 aa, Homo sapiens; identical to SEQ ID NO: 252

SEQ ID NO: 362 - DNA, 357 bp, Homo sapiens; identical to SEQ ID NO: 253

SEQ ID NO: 363 - protein, 109 aa, Homo sapiens; identical to SEQ ID NO: 254

SEQ ID NO: 364 - DNA, 327 bp, Homo sapiens; identical to SEQ ID NO: 255

SEQ ID NO: 365 - protein, 249 aa, Homo sapiens; identical to SEQ ID NO: 256

SEQ ID NO: 366 - DNA, 747 bp, Homo sapiens; identical to SEQ ID NO: 257

SEQ ID NO: 367 - protein, 10 aa, Homo sapiens
GYTFTTYGMN

SEQ ID NO: 368 - protein, 17 aa, Homo sapiens
WINTNTGKPTYAQGFTG

SEQ ID NO: 369 - protein, 10 aa, Homo sapiens
GYYGWDYHDY

SEQ ID NO: 370 - DNA, 30 bp, Homo sapiens; identical to SEQ ID NO: 261

SEQ ID NO: 371 - DNA, 51 bp, Homo sapiens; identical to SEQ ID NO: 262

SEQ ID NO: 372 - DNA, 30 bp, Homo sapiens; identical to SEQ ID NO: 263

SEQ ID NO: 373 - protein, 11 aa, Homo sapiens
GGNNIGSKSVH

SEQ ID NO: 374 - protein, 7 aa, Homo sapiens
YDSDRPS

SEQ ID NO: 375 - protein, 13 aa, Homo sapiens
QVWDSSSDHSPYV

SEQ ID NO: 376 - DNA, 33 bp, Homo sapiens; identical to SEQ ID NO: 267

SEQ ID NO: 377 - DNA, 21 bp, Homo sapiens
tatgatagcgaccggccctca

SEQ ID NO: 378 - DNA, 39 bp, Homo sapiens; identical to SEQ ID NO: 269

SEQ ID NO: 379 - protein, 119 aa, Homo sapiens; identical to SEQ ID NO: 270

SEQ ID NO: 380 - DNA, 357 bp, Homo sapiens; identical to SEQ ID NO: 271

SEQ ID NO: 381 - protein, 111 aa, Homo sapiens; identical to SEQ ID NO: 272

SEQ ID NO: 382 - DNA, 333 bp, Homo sapiens; identical to SEQ ID NO: 273

SEQ ID NO: 383 - protein, 251 aa, Homo sapiens; identical to SEQ ID NO: 274

SEQ ID NO: 384 - DNA, 753 bp, Homo sapiens; identical to SEQ ID NO: 275

SEQ ID NO: 385 - protein, 10 aa, Homo sapiens
GGTFSSYAIS

SEQ ID NO: 386 - protein, 17 aa, Homo sapiens
GIIPIFGTANYAQKFQG

SEQ ID NO: 387 - protein, 9 aa, Homo sapiens
WFYMQAGDH

SEQ ID NO: 388 - DNA, 30 bp, Homo sapiens; identical to SEQ ID NO: 5

SEQ ID NO: 389 - DNA, 51 bp, Homo sapiens; identical to SEQ ID NO: 6

SEQ ID NO: 390 - DNA, 27 bp, Homo sapiens
tggttctacatgcaggctggtgatcat

SEQ ID NO: 391 - protein, 14 aa, Homo sapiens
TGSSSDVGTYNYDS

SEQ ID NO: 392 - protein, 7 aa, Homo sapiens
DVSERPS

SEQ ID NO: 393 - protein, 10 aa, Homo sapiens
SSFAASSPWL

SEQ ID NO: 394 - DNA, 42 bp, Homo sapiens; identical to SEQ ID NO: 285

SEQ ID NO: 395 - DNA, 21 bp, Homo sapiens
gatgtcagtgagcggccctca

SEQ ID NO: 396 - DNA, 30 bp, Homo sapiens; identical to SEQ ID NO: 287

SEQ ID NO: 397 - protein, 118 aa, Homo sapiens; identical to SEQ ID NO: 288

SEQ ID NO: 398 - DNA, 354 bp, Homo sapiens; identical to SEQ ID NO: 289

SEQ ID NO: 399 - protein, 111 aa, Homo sapiens; identical to SEQ ID NO: 290

SEQ ID NO: 400 - DNA, 333 bp, Homo sapiens; identical to SEQ ID NO: 291

SEQ ID NO: 401 - protein, 250 aa, Homo sapiens; identical to SEQ ID NO: 292

SEQ ID NO: 402 - DNA, 750 bp, Homo sapiens; identical to SEQ ID NO: 293

SEQ ID NO: 403 - protein, 10 aa, Homo sapiens
GFTFSSYGMN

SEQ ID NO: 404 - protein, 17 aa, Homo sapiens
SISSSSSYIYYADSVKG

SEQ ID NO: 405 - protein, 14 aa, Homo sapiens
IQDATGEEMILYDY

SEQ ID NO: 406 - DNA, 30 bp, Homo sapiens; identical to SEQ ID NO: 297

SEQ ID NO: 407 - DNA, 51 bp, Homo sapiens; identical to SEQ ID NO: 298

SEQ ID NO: 408 - DNA, 42 bp, Homo sapiens; identical to SEQ ID NO: 299

SEQ ID NO: 409 - protein, 16 aa, Homo sapiens
RSSQSLVYSDGNTYLN

SEQ ID NO: 410 - protein, 7 aa, Homo sapiens
QVSKRDS

SEQ ID NO: 411 - protein, 8 aa, Homo sapiens
MQGSHLRT

SEQ ID NO: 412 - DNA, 48 bp, Homo sapiens; identical to SEQ ID NO: 303

SEQ ID NO: 413 - DNA, 21 bp, Homo sapiens
caggtttctaagcgggactct

SEQ ID NO: 414 - DNA, 24 bp, Homo sapiens
atgcaaggttcacacttgcggacg

SEQ ID NO: 415 - protein, 123 aa, Homo sapiens; identical to SEQ ID NO: 306

SEQ ID NO: 416 - DNA, 369 bp, Homo sapiens; identical to SEQ ID NO: 307

SEQ ID NO: 417 - protein, 112 aa, Homo sapiens; identical to SEQ ID NO: 308

SEQ ID NO: 418 - DNA, 333 bp, Homo sapiens; identical to SEQ ID NO: 309

SEQ ID NO: 419 - protein, 256 aa, Homo sapiens; identical to SEQ ID NO: 310

SEQ ID NO: 420 - DNA, 768 bp, Homo sapiens; identical to SEQ ID NO: 311

SEQ ID NO: 421 - protein, 10 aa, Homo sapiens
GYTFTDYYIH

SEQ ID NO: 422 - protein, 17 aa, Homo sapiens
WMNPNSGNSVSAQKFQG

SEQ ID NO: 423 - protein, 14 aa, Homo sapiens
YQGSTWKYDSYGDL

SEQ ID NO: 424 - DNA, 30 bp, Homo sapiens; identical to SEQ ID NO: 315

SEQ ID NO: 425 - DNA, 51 bp, Homo sapiens; identical to SEQ ID NO: 316

SEQ ID NO: 426 - DNA, 42 bp, Homo sapiens; identical to SEQ ID NO: 317

SEQ ID NO: 427 - protein, 11 aa, Homo sapiens
GGNEIGFNGVH

SEQ ID NO: 428 - protein, 7 aa, Homo sapiens
NNRVRPS

SEQ ID NO: 429 - protein, 11 aa, Homo sapiens
QVWVNPDNEYV

SEQ ID NO: 430 - DNA, 33 bp, Homo sapiens; identical to SEQ ID NO: 321

SEQ ID NO: 431 - DNA, 21 bp, Homo sapiens
aacaatagggtccggccctca

SEQ ID NO: 432 - DNA, 33 bp, Homo sapiens; identical to SEQ ID NO: 323

SEQ ID NO: 433 - protein, 123 aa, Homo sapiens; identical to SEQ ID NO: 324

SEQ ID NO: 434 - DNA, 369 bp, Homo sapiens; identical to SEQ ID NO: 325

SEQ ID NO: 435 - protein, 109 aa, Homo sapiens; identical to SEQ ID NO: 326

SEQ ID NO: 436 - DNA, 327 bp, Homo sapiens; identical to SEQ ID NO: 327

SEQ ID NO: 437 - protein, 253 aa, Homo sapiens; identical to SEQ ID NO: 328

SEQ ID NO: 438 - DNA, 759 bp, Homo sapiens; identical to SEQ ID NO: 329

SEQ ID NO: 439 - protein, 10 aa, Homo sapiens
GFTFSSYEMN

SEQ ID NO: 440 - protein, 17 aa, Homo sapiens
YISSSGSTIYYADSVKG

SEQ ID NO: 441 - protein, 13 aa, Homo sapiens
DWRSSYYYSQYDK

SEQ ID NO: 442 - DNA, 30 bp, Homo sapiens; identical to SEQ ID NO: 333

SEQ ID NO: 443 - DNA, 51 bp, Homo sapiens; identical to SEQ ID NO: 334

SEQ ID NO: 444 - DNA, 39 bp, Homo sapiens; identical to SEQ ID NO: 335

SEQ ID NO: 445 - protein, 13 aa, Homo sapiens
TRSSGNIASNYVQ

SEQ ID NO: 446 - protein, 7 aa, Homo sapiens
ADNQRPS

SEQ ID NO: 447 - protein, 9 aa, Homo sapiens
QSYENNIHV

SEQ ID NO: 448 - DNA, 39 bp, Homo sapiens; identical to SEQ ID NO: 339

SEQ ID NO: 449 - DNA, 21 bp, Homo sapiens
gcggacaaccaaagaccctct

SEQ ID NO: 450 - DNA, 27 bp, Homo sapiens
cagtcttatgaaaacaacattcacgtg

SEQ ID NO: 451 - protein, 122 aa, Homo sapiens; identical to SEQ ID NO: 342

SEQ ID NO: 452 - DNA, 366 bp, Homo sapiens; identical to SEQ ID NO: 343

SEQ ID NO: 453 - protein, 111 aa, Homo sapiens; identical to SEQ ID NO: 344

SEQ ID NO: 454 - DNA, 333 bp, Homo sapiens; identical to SEQ ID NO: 345

SEQ ID NO: 455 - protein, 254 aa, Homo sapiens; identical to SEQ ID NO: 346

SEQ ID NO: 456 - DNA, 762 bp, Homo sapiens; identical to SEQ ID NO: 347